(19) United States
(12) Patent Application Publication
(10) Pub. No.: US 2002/0071824 A1
(43) Pub. Date: Jun. 13, 2002

(54) GENE TRANSFER TO PANCREATIC B CELLS FOR PREVENTION OF ISLET DYSFUNCTION

(76) Inventors: NICK GIANNOUKAKIS, PITTSBURGH, PA (US); PAUL D. ROBBINS, MY. LEBANON, PA (US); MASSIMO TRUCCO, PITTSBURGH, PA (US)

Correspondence Address: BAKER & BOTTS, 30 ROCKEFELLER PLAZA, NEW YORK, NY, 10112

(21) Appl. No.: 09/320,767

(22) Filed: May 27, 1999

(51) Int. Cl. A61K 48/00; C12Q 1/68; C07H 21/04

(52) U.S. Cl. 424/85.1; 514/44; 435/6; 435/91.1; 435/375; 435/325; 435/455; 435/320.1; 536/23.1; 536/24.31; 536/24.33; 536/24.5

(57) ABSTRACT

The present invention relates to methods and compositions for inhibiting pancreatic β cell dysfunction and Fas-mediated apoptosis. The invention relates to recombinant vectors, including viral vectors, comprising nucleic acids molecules encoding inhibitors of interleukin-1β (IL-1β) and Fas-mediated apoptosis and the use of such vectors for transfer of said nucleic acid molecules into β cells. The invention encompasses genetically engineered β cells comprising nucleic acid molecules encoding inhibitors of IL-1β signal transduction. The invention further relates to methods for transplanting such genetically engineered β cells into a host recipient with a pancreatic disorder. The methods and compositions of the invention may be used to reduce Il-1β mediated β cell dysfunction and apoptosis, thereby reducing the insulitis associated with pancreatic disorders such as insulin dependent diabetes mellitus (IDDM).

### INTRODUCTION

The present invention relates to methods and compositions for inhibiting pancreatic β cell dysfunction and Fas-mediated apoptosis. The invention relates to recombinant vectors, including viral vectors, comprising nucleic acids molecules encoding inhibitors of interleukin-1β (IL-1β) and Fas-mediated apoptosis and the use of such vectors for transfer of said nucleic acid molecules into β cells. The invention encompasses genetically engineered β cells comprising nucleic acid molecules encoding inhibitors of IL-1 β signal transduction. The invention further relates to methods for transplanting such genetically engineered β cells into a host recipient with a pancreatic disorder. The invention is based on the observation that transfer of nucleic acid molecules encoding inhibitors of IL-1β, such as the interleukin-1 receptor antagonist protein (IL-1Ra) or insulin like growth factor-1 (IGF-1), into β cells reduces β cell dysfunction and Fas-mediated apoptosis. The methods and compositions of the invention may be used to reduce Il-1β mediated β cell dysfunction and apoptosis, thereby reducing the insulitis associated with pancreatic disorders such as insulin dependent diabetes mellitus (IDDM).

### BACKGROUND OF INVENTION

Insulin-dependent diabetes mellitus (IDDM) is characterized by a local inflammatory reaction in and around the islets of Langerhans followed by selective destruction of pancreatic islet β cells. In both humans and the non-obese diabetic (NOD) mouse, the immunopathology is characterized by an early-onset insulitis with a significant proportion of the invading cells consisting of T-lymphocytes which may directly damage β cells by secreting proinflammatory interleukins (Bach J. F., 1994, Endocr Rev 15:516; Tisch R. and McDevitt H. 1996, Cell 85:291; Brennan F. M. et al., 1992, Curr Opin Immunol 4:754). Immunohistochemistry and morphology have demonstrated an increase in infiltrating macrophages just before, and at onset of insulitis in the NOD mouse (Reddy S. et al., 1993, Pancreas 8:602). Thus, resident macrophages have also been proposed to be another cell type that may initiate β cell damage during development of autoimmune diabetes.

Interleukin-1β (IL-1β) is a proinflammatory cytokine produced in response to infection, toxic injury, trauma and antigenic stimulation and which has pleiotropic effects on many cell types including β cells. The major source of IL-1 is the macrophage and its expression can be induced by IFN-gamma, although B-lymphocytes, endothelial, mesengial, smooth muscle cells and fibroblasts can produce IL-1β as well. Two different genes encode IL-1 resulting in two peptides, IL-1α and IL-1β. The IL-1β is the more abundant of the mRNAs and is the major secreted form of IL-1. IL-1 binds to two cell surface receptors, referred to as the type I receptor, which transmits its biological signal, and the type II receptor which is not known to transduce any signal and may act as a decoy molecule to down regulate IL-1 induced inflammation.

IL-1β has been shown to be the initiating cytokine that is directly responsible for the impairment of glucose-stimulated insulin production in mouse, rat and human islets in vitro (McDaniel M. L. et al., 1996, Proc. Soc. Exp Biol Med 211:24; Corbett J. A. et al., 1993, Autoimmunity 15:145; Corbett J. A. et al., 1992, J. Clin Invest 90:2384). IL-1β also induces the expression and enhances the activity of the inducible form of nitric oxide synthase resulting in elevated levels of nitric oxide (Arnush M. et al., 1998, J. Immunol 160:2684; Arnush M. et al., 1998, J. Clin Invest 102:516; McDaniel M. L. et al., 1996, Proc. Soc. Exp Biol Med 211:24; Corbett J. A. et al., 1993, Autoimmunity 15:145; Corbett J. A. et al., 1992, J. Clin Invest 90:2384).

It is thought that nitric oxide is a direct mediator of IL-1β-stimulated impairment of glucose-stimulated insulin secretion by islets and single dispersed islet cells in vitro. A series of studies using L-NMMA, an inhibitor of inducible nitric oxide synthase (iNOS), were shown to prevent IL- 1β-induced impairment of glucose-stimulated insulin production (Corbett J. A. et al., 1992, Biochem J 287:229; Corbett J. A. et al., 1994 Biochem J 299:719). In addition, an IL-1β-stimulated decrease of cell viability in vivo in rats was inhibited following the administration of chemical inhibitors of nitric oxide synthase (Bolaffi J. L. et al., 1994, Endocrinology 134:537).

Recent observations indicate that a substantial amount of the β cell death may also be due to Fas-mediated apoptosis. The Fas antigen (CD95/APO-1) is a transmembrane molecule that belongs to the TNF family of receptor proteins and is involved in the induction of apoptosis of a wide variety of cells (Nagata S. et al., 1995, Immunol Today 16:39). Several different lines of evidence indicate that apoptosis in NOD mice occurs as early as 3 weeks of age and is mediated through Fas-FasL interaction. In particular, T cells are able to induce apoptosis of β cells through direct interaction of FasL on T cells with Fas expressed specifically on β cells.

IL-1β also stimulates the cell surface expression, in murine and human islets, of Fas and evidence indicates that it may be NO-dependent (Yamuda K et al., 1996, Diabetologia 39:1306; Stussi G et al., 1997, J. Exp Med 186:1193). Normal human pancreatic β cells, that do not constitutively express Fas, become strongly Fas-positive after IL-1β exposure, and then become susceptible to Fas-mediated apoptosis. N^{g}-monomethyl-L-arginine, an inhibitor of nitric oxide (NO) synthase, prevents Il-1β induced functional Fas expression in normal pancreatic β cells. The selective expression of Fas in β cells primed by NO may be responsible for their specific killing since T cells expressing FasL may promote an MHC-unrestricted destruction of the Fas positive β cells, while sparing neighboring Fas negative α and δ cells (Stassi G et al., 1997, J Exp Med 186:1193).

Earlier studies showed that cytokines such as TNFα and IFNγ could also impair β cell function, but more recent data strongly indicate that these cytokines act on resident macrophages to produce IL-1β which can then bind the signaling type I IL-1 receptor that is expressed on β cells and mediate the IL-1β signal (Lacy P. E. et al., 1991, Am J Pathol 138:1183; Lacy P. E., 1994, Mt. Sinai J. Med. 61:170; Scarim A. L. et al., 1997, Biochem Biophys Acta 1361:313). Blocking IL-1β using an antibody prevented the induction of insulitis and diabetes in cyclophosphamide-induced diabetic NOD mice (Caillcan C. et al., 1997, Diabetes 46:937).

A naturally occurring inhibitor of IL-1β action is the interleukin-1 receptor antagonist protein (IL-1Ra). It binds to the type I IL-1 receptor, which transmits the biologic actions of IL-1, in a competitive manner but does not bind the type II receptor, believed to act as a scavenger of IL-1β (Dripps D. J. et al., 1991, J. Biol Chem 266:10331; Grunowitz E. V. et al., 1992, Blood 79:2364; Symons J. A., 1995, Proc. Natl. Acad Sci USA 92:1714; Thompson, R. C., 1991, Agents Actions Suppl. 35:41; Dinarello C. A., Kidney Int. Suppl. 1992 38:568; Dinarello C. A., 1992, Eur Cytokine Netw 3:7; Dripps D. J., 1991, J. Biol Chem. 266:20311). IL-1Ra was shown to be effective, in recombinant form, in suppressing NO production and the IL-1β stimulated and NO-mediated suppression of insulin production following a glucose challenge to islets and purified β cells in vitro (Arnush M. et al., 1998, J. Clin Invest 102:516; Scarim A. L. et al., 1997, Biochem Biophys Acta 1361:313). In vivo, recombinant IL-1Ra given as a continuous infusion into NOD mice, prevented the loss of syngeneic islet transplants. (Sandbery J. D. et al., 1997, J Virol 71:1842).

β cell development and survival is in part controlled by the availability of growth factors and in particular the insulin-like growth factors (IGFs) (Hill D. J. and Hogg J. 1991, Baillieres Clin. Endocinol Metab 5:689-98; Hill D. J. and Hogg J. 1992, Adv. Exp. Med. Biol. 321: 113-20; Hill D. J. et al., 1987, Diabetes 36:465-471; Hill, D. J. et al., 1998, Diabetes Care 21 Suppl 2 B60-9). The IGFs consist of two potent mitogens, IGF-II and IGF-I. The former plays a prominent role during fetal growth in a wide variety of mammals including humans and is expressed in many tissues postnatally, albeit at lower levels. IGF-I expression progressively increases in liver during development to ultimately mediate the growth-promoting effects of growth hormone postnatally. Although the liver is the major site of IGF-I synthesis, many other tissues are capable of secreting IGF-I in a growth-hormone-independent manner. In these tissues, IGF-I acts in a paracrine and/or autocrine fashion and its actions are modulated by six characterized binding proteins (Jones, J. I., 1995, Endocr Rev 16: 3-34).

The predominant site of IGF production in rodent and human islets is the β cell, which can also respond to IGF through expression of the type I IGF signaling receptor. There is good evidence that IGFs act in an autocrine and paracrine manner during islet development as mitogens and they may modulate insulin release postnatally, directly or indirectly through changes in β cell mass or glucose responsiveness. Human fetal pancreas contains IGF-I and appears to secrete IGF-I in a glucose-dependent manner in vitro. In addition to their growth-promoting effects, the IGFs, and IGF-I in particular, display anti-apoptotic characteristics, in hematopoietic and myeloid progenitor cells, neurons, cardiomyocytes, and a variety of tumor cell lines (Russell, J. W. et al., 1998, J Neurobiology 36: 455-67).

Petrik et al. have recently shown that apoptosis in islets may be developmentally-regulated and that the peak of apoptosis coincides with low IGF-II and increased iNOS levels. Incubation of islets during the period of peak apoptosis in the presence of IGF-I or IGF-II was able to increase the rate of β cell survival (Petrik J. et al, 1998, Endocrinology 139:2994). Neonatal rat islets cultured with IL-1β, TNFα and IFNγ, underwent rapid apoptosis when co-incubated with an agonistic anti-Fas antibody (Harrison, M. et al., 1998, FEBS Lett 435:207-10). Preincubation with IGF-I, however, prevented IL-1β-induced, Fas-triggered apoptosis. In a similar study, pretreatment of rat islets with recombinant IGF-I, resulted in a decrease of IL-1β-mediated NO formation by inhibition of iNOS expression and synthesis as well as restoration of glucose-stimulated insulin release (Mabley J. G. et al., 1997, FEBS Lett 417:235). Additionally, IGF-I treatment of early age NOD mice could decrease the incidence of IDDM as well as the insulitis grade (Kaino Y. et al., 1996, Diabetes Res Clin Pract 34:7-10). Recombinant IGF-I administration to NOD mice was able to promote the integrity of β cell mass as well as reduce the incidence of adoptive transfer of IDDM with T cells from diabetic NOD mice (Bergerot I et al., 1995, Clin Exp Immunol 102: 335-40; Bergerot I et al., 1996, Diabetes Metab 22:235-9).

Islet transplantation is a viable therapy for type I diabetes, however, the allogenic response to donor antigens makes graft acceptance an important obstacle. Recently, the use of immunosuppressive cytokines or proteins that can block the allogenic response have yielded promising results where allogenic and even xenographic graft survival was prolonged following engraftment. The direct transfer of genes encoding immunosuppressive agents to islets could be used to prolong engraftment of allogenic islets following transplantation. A number of groups have successfully used viral-mediated gene transfer of immunoregulatory genes such as IL-10, viral IL-10, CTLA4-Ig to islets in vitro and in vivo, mostly employing E1-deleted, first-generation adenoviral vectors (Csete M. E. et al., 1995, Transplantation 59: 263-8; Csete M. E. et al., 1994, Transplant Proc 26:756-7; Benhamon P. Y., et al., 1996, Transplantation 62:1306-12; Gainer A. L. et al., 1997, Transplantation 63:1017; Gainer A. L. et al., 1998, Transplant Proc. 30:534; Guo, Z et al. 1998, Transplant Proc 30: 589).

E1-deleted adenoviral vectors have been tested in human islets extensively and they do not appear to interfere with the function of the β cell in vitro (Weber M. et al., 1997, J. Surg Res 69:23-32). In vivo however, the inherent immunogenicity of these vehicles may be detrimental to the islet transplant, at least as it pertains to a gene therapy based treatment of IDDM, where the virus and its proteins could induce a virus specific immune response.

### 3. SUMMARY OF THE INVENTION

The present invention relates to compositions and methods for inhibiting IL-1β mediated β cell dysfunction and apoptosis. The compositions of the invention encompass recombinant nucleic acid molecules, including expression vectors, encoding inhibitors of IL-1β activity and genetically engineered β cells expressing such nucleic acid molecules.

The invention further provides methods wherein the recombinant nucleic acid molecules encoding inhibitors of IL-1β activity are transferred into β cells. Delivery of a nucleic acid molecule encoding an IL-1β inhibitor into a host cell may be either direct, in which case the host is directly exposed to the nucleic acid or nucleic acid-carrying vector, or indirect, in which case, host cells are first transformed with the nucleic acid in vitro, then transplanted into the host. The transfer of such nucleic acid molecules may be accomplished using various delivery systems including but not limited to encapsulation of the nucleic acid molecules into liposomes and microparticles, or the use of recombinant vectors genetically engineered to express proteins capable of inhibiting β cell dysfunction and apoptosis.

The present invention further provides for the transplantation of genetically engineered β cells, expressing IL-1β inhibitors, into a host recipient to reduce β cell dysfunction and apoptosis associated with pancreatic disorders. The β cells may also be genetically engineered to express molecules, such as immunosuppressive molecules, to suppress rejection of the transplanted cells and improve graft survival.

The present invention is based on the observation that transfer of nucleic acid molecules encoding IL-1Ra or IGF-1 into pancreatic β cells inhibits β cell dysfunction and apoptosis. The methods and compositions of the invention may be used to reduce β cell dysfunction and apoptosis associated with pancreatic disorders such as insulin dependent diabetes mellitus (IDDM).

### 4. BRIEF DESCRIPTION OF THE DRAWINGS

FIG. 1A. IL-1β actions on β cells can be regulated at three levels: (1) at the level of receptor binding; (2) at the post-receptor level; and (3) at the nuclear signaling level.

FIG. 1B. IL-1β stimulated β cell Fas-mediated apoptosis. IL-1β stimulates the expression of Fas on the surface of the β cells which binds to FasL expressed on activated T cells.

FIG. 1C. The presence of the interleukin-1 receptor antagonist protein (IRAP) blocks IL-1β induced signal transduction and Fas-mediated apoptosis in β cells.

FIG. 2. Reporter gene expression in adenovirally-infected human islets in vitro. Panel I: Ad-eGFP-infected islets are the intensely fluorescent spherical cellular aggregates (×200 magnification), Panel II: Ad-LacZ-infected islet is the X-gal-positive spherical cell aggregate in the middle of the panel (×200 magnification), Panel III: mock-infected islets visualized under fluorescence microscopy (×20 magnification). Besides intact islets, single islet cells and fibroblasts are also infected by adenoviral vectors.

FIG. 3. Glucose-stimulated insulin release from human islets infected with Ad-LacZ, Ad-IL-1Ra, or mock-infected followed by an incubation with or without IL-1β. Bars indicate the means of three separate experiments each performed in triplicate and represent percent above control, where the production of insulin by untreated, uninfected islets in the presence of 5 mM glucose is taken as control (100%). The error bars indicate the SEM.

FIG. 4. Nitric oxide production by human islets infected with Ad-IL-1Ra in vitro. Bars indicate the means of three separate experiments each performed in triplicate (expressed as percent of control, where the nitrite level in uninfected, control islets is taken as 100%) and the error bars indicate the SEM.

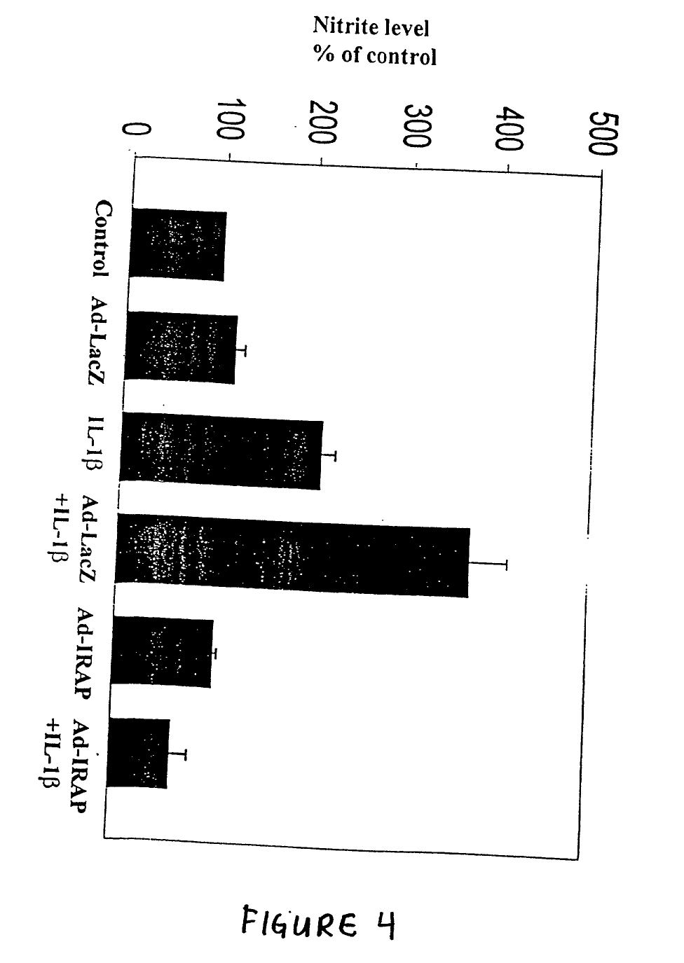

FIG. 5. Caspase-3 activity in human islets infected with Ad-IL-1Ra in vitro. Bars indicate the means of three separate experiments each performed in triplicate. The values are expressed as percent of control, where the mean of the Caspase-3 activity to DNA content ratio in uninfected, untreated control islets is taken as 100%.

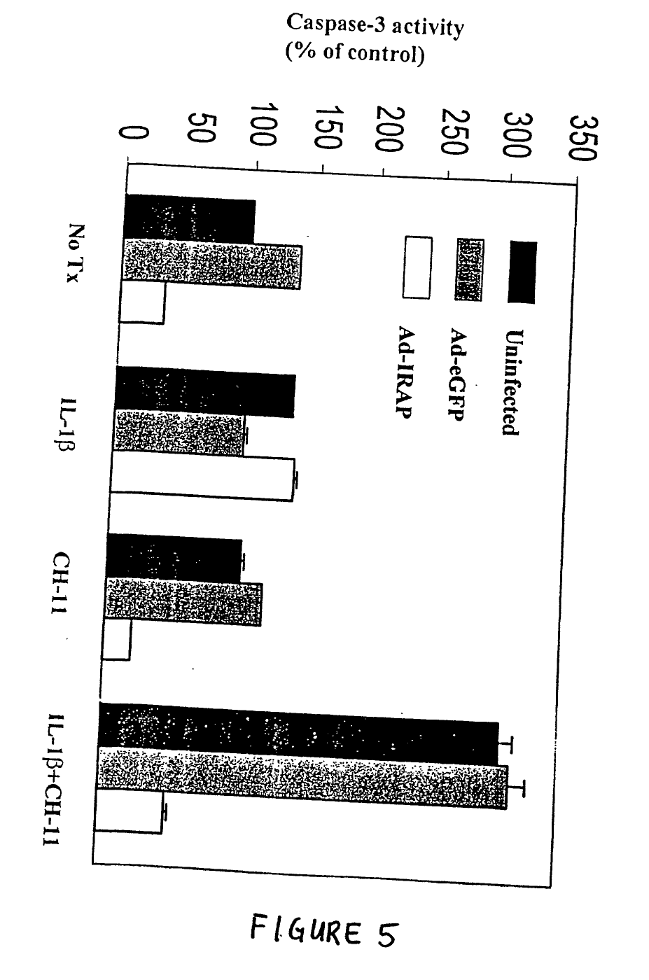

FIG. 6. Efficient eGFP reporter gene expression in human islets following adenoviral gene transfer. In panel A are mock infected islets (×20 magnification) and in panel B are Ad-eGFP-infected islets (×200 magnification).

FIG. 7. Adenoviral gene transfer of IGF-I to human islets prevents the IL-1β-induced impairment of glucose-stimulated insulin release. The bars indicate the means of triplicate experiments with triplicate determinations each and the error bars denote the SEM. The data is presented as percent above control where the insulin secretion by untreated, uninfected islets exposed to 5 mM glucose is taken as 100%.

FIG. 8. Adenoviral gene transfer of IGF-I to human islets can prevent IL-1β-induced nitric oxide production. Bars indicate the means of three different experiments each performed in triplicate and the error bars denote the SEM. Values are shown as percent of control where the nitrite level in mock-infected, untreated islets is taken as 100%.

FIG. 9. Infection of human islets in culture with an adenoviral vector expressing human IGF-I can suppress IL-1β-stimulated, Fas-triggered activation of apoptosis. The bars indicate the means of three independent experiments performed in triplicate and the error bars denote the SEM. We show the means as percent of control where the ratio of caspase-3 activity to DNA content in uninfected, untreated islets represents 100%.

FIG. 10. β galactosidase expression in human islets following lentiviral and adenoviral delivery in vitro. Human islets were infected with 2×10⁶ pfu of Ad-LacZ or an identical TIU of LtV-LacZ. Panel A shows uninfected islets exposed to X-gal. Panel B shows X-gal staining of islets infected with Ad-LacZ and Panel C shows X-gal staining of islets infected with LtV-LacZ (200×magnification).

FIG. 11A. eGFP expression in single dispersed islet cells derived from intact islets infected with Ad-eGFP or LtV-eGFP. Panel I illustrates islet cells obtained from islets infected with Ad-eGFP and Panel II shows islet cells derived from islets infected with LtV-eGFP. Panel III shows that single cells derived from uninfected islets do not exhibit any fluorescence.(40×magnification)

FIG. 11B. FACS analysis of dispersed islet cells expressing enhanced green fluorescent protein and gated for scatter between 1-15°. Fluorescence of uninfected islet cells correspond to the peak on the far left. The area underneath the arrows represents the fluorescence of islet cells derived from islets infected by LtV-eGFP (solid black line) and the fluorescence of cells derived from islets infected by AD-eGFP (shaded grey area).

FIG. 11C. FACS analysis of dispersed single islet cells which are eGFP-positive. There is no significant difference between the number of gated cells that are eGFP positive (those with scatter between 1-15°) derived from islets infected with an equal number of viral particles of LtV-eGFP or Ad-eGFP. Note that none of the uninfected cells expressing eGFP. Bars indicate the means of triplicate determinations and error bars show the SEM.

FIG. 12. Human islets infected with LtV-IL-1Ra produce human IL-1Ra in the culture media. Bars indicate the values of independent determinations.

FIG. 13. Glucose-stimulated insulin release into the culture buffer of islets infected with LtV-eGFP or with Ad-eGFP. Bars indicate the means of triplicate determinations and error bars denote the SEM

### 5. DETAILED DESCRIPTION OF THE INVENTION

The present invention relates to methods and compositions for inhibiting IL-1β mediated β cell dysfunction and Fas-mediated apoptosis. As described in detail below the invention encompasses genetically engineered vectors including viral vectors comprising nucleic acid molecules encoding inhibitors of IL-1β activity and Fas-mediated apoptosis and the transfer of said nucleic acid molecules into pancreatic β cells. Further, the invention relates to compositions which include genetically engineered pancreatic β cells comprising nucleic acid molecules encoding inhibitors of IL-1β activity. Such cells may be transplanted into a host recipient to reduce or eliminate the symptoms of insulin dependent diabetes mellitus associated with β cell dysfunction and apoptosis.

### 5.1. Inhibitors of IL-1β Mediated β Cell Dysfunction and Apoptosis

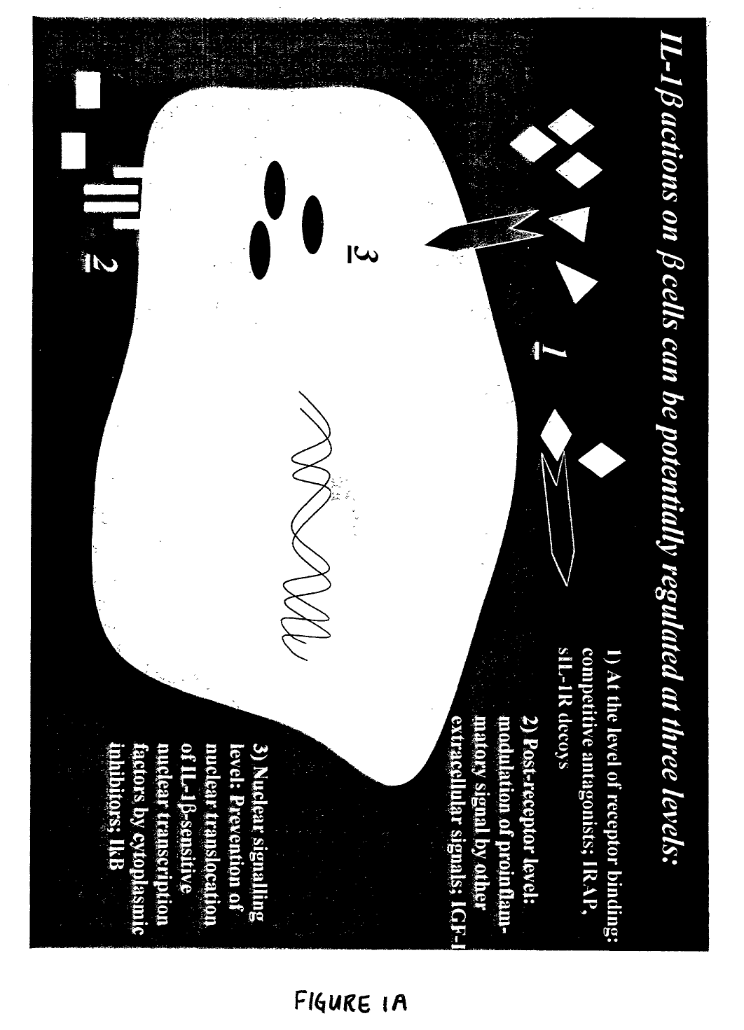

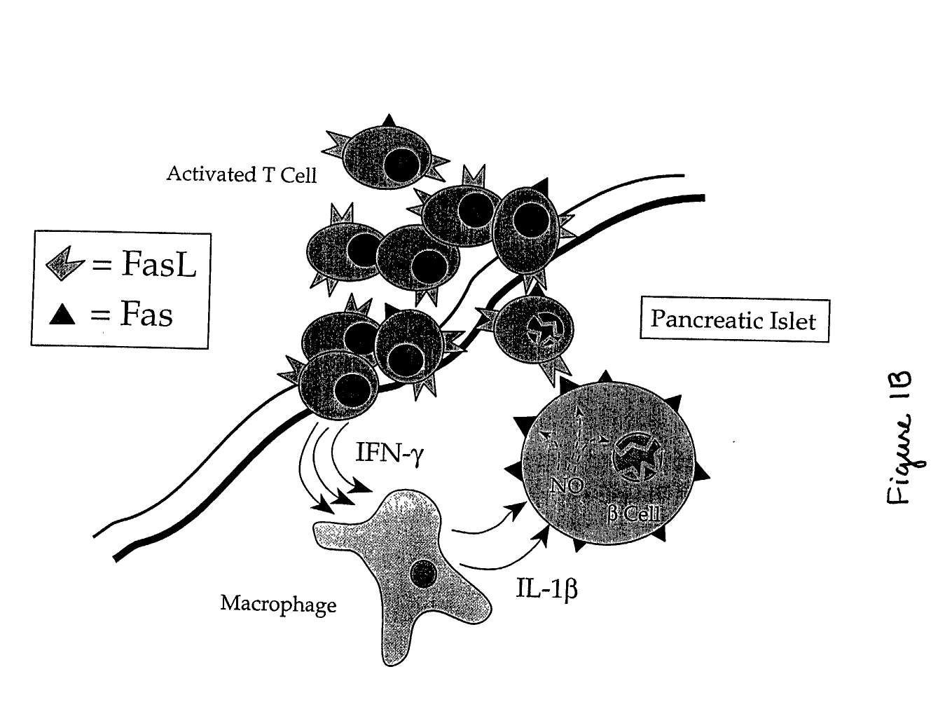

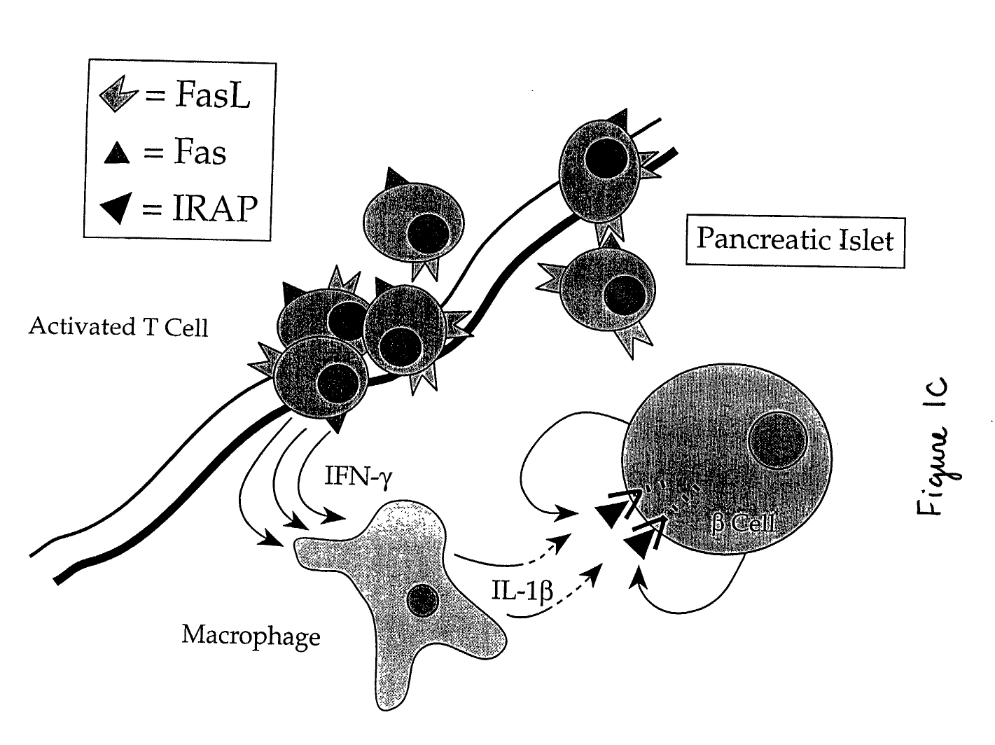

The present invention relates to recombinant nucleic acid molecules encoding inhibitors of IL-1β activity and the transfer of said nucleic acid molecules into β cells to reduce IL-1β mediated cell dysfunction and Fas-mediated apoptosis (see, FIG. 1A). Inhibitors of IL-1β activity include molecules that interact with the IL-1β receptor and inhibit the activity triggered by IL-1β, as well as molecules that bind to and neutralize IL-1β. Other inhibitors include those that affect the expression or activity of a factor, such as intracellular factors, involved in the IL-1β signal transduction pathway. Additionally, recombinant nucleic acid molecules encoding inhibitors of Fas-mediated apoptosis may be transferred into β cells to reduce the level of apoptosis.

The present invention is based on the discovery that transfer of nucleic acid molecules encoding the IL-1Ra protein to cultured islets results in protection of the islets against IL-1β induced nitric oxide formation, impairment in glucose-stimulated insulin production and Fas-triggered apoptosis. Additionally, the transfer of nucleic acid molecules encoding IGF-1 into cultured islet cells prevents IL-1β induced nitric oxide formation and impairment of glucose-stimulated insulin production. IGF-1 gene transfer also prevented IL-1β induced apoptosis as detected by a decrease in Fas-triggered caspase-3 activity.

In an embodiment of the invention, the activity of IL-1β can be regulated at the level of receptor binding using, for example, nucleic acid molecules encoding competitive antagonists of IL-1β. Such antagonists include but are not limited to the naturally occurring interleukin-1 receptor antagonist protein (IL-1Ra;also referred to as IRAP), soluble interleukin-1 receptor “decoys” that are capable of competing with wild type IL-1β for receptor binding but which fail to activate the signaling activity of the receptor and soluble type I tumor necrosis factor alpha receptors (Ghivizzani S C et al., 1998, Proc. Natl. Acad. Sci USA 95:4613-8). Soluble interleukin I receptors that can be used include those described in U.S. Pat. Nos. 5,319,071, 5,767,065 and RE 35,450. Additionally, vaccina and cowpox virus encode a soluble form of type II IL-IR that can compete for IL-1 binding to cell surface receptors and thus reduce IL-1 activity (Barry M., 1998, Curr Opin Immunol 10:422-30).

Alternatively, the activity of IL-1β can be regulated utilizing nucleic acid molecules encoding modulators of the pro-inflammatory signal induced by IL-1β. For example, nucleic acids encoding cytokines such as insulin growth factor-I (IGF-1) can be used to reduce the β cell dysfunction and apoptosis mediated by activation of the IL-1β signal transduction pathway. Other such molecules include but are not limited to nucleic acid molecules encoding IGF-II.

Stimulation of the IL-1β signal transduction pathway results in activation of nuclear transcription factors such as AP1 (fos/jun) and NF-κB, and these in turn activate IL-1 responsive genes. For example, IL-1β mediated induction of nitric oxide synthase expression occurs via translocation of the transcription factor NF-κB from the cytoplasm into the nucleus. Thus, nucleic acid molecules encoding inhibitors of IL-1β mediated nuclear signaling can be used to antagonize the activity of IL-1β. Such inhibitors include NF-κB inhibitors, STATS, STAT6 and NF-AT (LaCasse EC et al., 1998 Oncogene 17:3247-59).

IL-1β induces Fas (CD95) upregulation on β cells and FasL triggered apoptosis. In an embodiment of the invention, nucleic acid molecules encoding direct inhibitors of Fas-mediated apoptosis may be used to reduce β cell dysfunction. Such inhibitors may include but are not limited to nucleic acid molecules encoding signaling-defective or dominant negative variants of Fas (Cascino I et al, (1996, J Immunol 156:13-17: Fisher GH et al., 1995, Cell 81:935-46); mutant or dominant negative mutants of downstream effectors of Fas, such as the Fas-associated death domain protein (FADD) (Wajant, H. et al., 1998, Curr. Biol 15:113-6: Newton K., et al., 1998, EMBO J 17:706-18:Chinnaiyan A. M., 1996, J. Biol. Chem 271:4961-5); inhibitors of the proapoptotic cascade such as the cowpox crmA protein (Marsters et al., 1998, Curr Biol 6:750-2; Barry M et al., 1998, Curr Opin Immunol 10:422-30) and members of the bcl-2 family including but not limited to Bcl-2 and Bcl-XL (Tsujimoto Y. et al., 1998, Genes Cells 3:697-707;Reed, 1998, Oncogene 17:3225-36; Adams et al., 1998, Science 281:1322-6:Kelkar et al., 1998, Trends Cell Biol 8:324-30).

In addition, inhibitors of IL-1β that may be utilized for purposes of the present invention include any additional IL-1β inhibitors identified using routine methods well known to those skilled in the art. For example, assays for identifying inhibitors of IL-1β activity include those designed to assay the response of β cells to glucose challenge as measured by a decrease in insulin release in the presence of IL-1β. Nitric oxide production may also be evaluated using, for example, a Griess reaction, wherein a reduction in nitric oxide production in the presence of IL-1β indicates the identification of an IL-1β inhibitor.

Further, a variety of different assays developed to measure apoptosis may be utilized to identify inhibitors of Fas-mediated apoptosis. After exposure to IL-1β, the β cells can be assayed for inhibition of apoptosis using assays which include: terminal deoxynucleotidly transferase mediated dUTP nick end labeling (TUNEL) assays (Kebers et al., 1998, Experimental Cell Research 240:197-205); assays to detect activated caspases (Janicke et al., 1998, J. Biol. Chem. 273:9357-9360); DNA ladder gel assays to detect fragmented DNA by gel electrophoresis (Bursch et al., 1996, Carcinogenesis 17:1595-1607); assays to detect bcl-2 and bax protein levels (Wuerzberger et al., 1998, Cancer Research 58:1876-1885); Hoechst/DAPI staining to detect nuclear condensation in apoptotic cells (Bursch et al., 1998, Carcinogenesis 17:1595-1607); Annexin V staining of phospatidyl serine on the cytoplasmic membrane (van Engeland et al., 1996, Cytometry 24:131-139); analysis of DNA content by propidium iodide staining followed by flow cytometry (Sherwood et al., Methods in Cell Biology 46:77-97; and morphological studies using electron and phase contrast microscopy (Bursch et al., Carcinogenesis 17:1595-1607); Annexin V staining of phospatidyl serine on the cytoplasmic membrane (van Engeland et al., 1996, Cytometry 24:131-139); and analysis of DNA content by propidium iodide staining followed by flow cytometry (Sherwood et al., Methods in Cell Biology 46:77-97).

### 5.2. Nucleic Acid Molecules Encoding Inhibitors of Apoptosis

The present invention relates to the transfer of nucleic acid molecules encoding biologically active proteins capable of inhibiting IL-1β into β cells. Such nucleic acid molecules include but are not limited to those encoding IL-1Ra, NF-Kβ inhibitor, AP1 inhibitor, sluble forms of the IL-1 R, mutant forms of the fas or FADD protein, IGF-1, the cowpox crmA protein, members of the bcl-2 family such as Bcl-2 and Bcl-XL. The nucleic acid molecules encoding such proteins are known in the art and can be isolated from a variety of different sources including but not limited to vertebrate, mammalian and human sources without undue experimentation, by molecular biological techniques well known in the art. For example, the DNA may be obtained from cloned DNA (e.g., a DNA “library”), by chemical synthesis, by cDNA cloning, or by the cloning of genomic DNA, or fragments thereof, purified from the desired cell (See, for example, Sambrook et al., 1989, Molecular Cloning, A Laboratory Manual, 2d Ed., Cold Spring Harbor Laboratory Press, Cold Spring Harbor, N.Y. Glover, D. M. (ed.), 1985, DNA Cloning: A Practical Approach MRL Press, Ltd., Oxford, U.K. Vol. I, II.).

Alternatively, polymerase chain reaction (PCR) may be used to amplify the desired nucleic acid sequence in a genomic or cDNA library. In such instances, oligonucleotide primers representing known nucleic acid sequences within the desired nucleic acid sequence can be used as oligonucleotide primers in PCR. The synthetic oligonucleotides may be utilized as primers to amplify by PCR sequences from a source (RNA or DNA), preferably a cDNA library. PCR can be carried out, e.g., by use of a Perkin-Elmer Cetus thermal cycler and Taq polymerase (Gene Amp).

Recombinant DNA cloning techniques well known in the art for expressing a nucleic acid molecule can be used to express an inhibitor of IL-1β activity. Such methods can be used to construct expression vectors containing nucleic acid molecules encoding IL-1β inhibitors and appropriate transcriptional and translational control signals. See, for example, the techniques described in Sambrook et al., 1989, supra, and Ausubel et al, 1989, supra.

Expression vectors can be plasmid, viral, or others known in the art, used for replication and expression in mammalian cells. Expression of the sequence encoding the inhibitor of apoptosis can be regulated by any promoter known in the art to act in mammalian, preferably human cells. Such promoters can be inducible or constitutive. Such promoters include but are not limited to: the SV40 early promoter region (Benoist, C. and Chambon, P. 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:14411445), the regulatory sequences of the metallothionein gene (Brinster et al., 1982, Nature 296:39-42), the viral CMV promoter, the human chorionic gonadotropin-β promoter (Hollenberg et al., 1994, Mol. Cell. Endocrinology 106:111-119), etc. Any type of plasmid, cosmid, YAC or viral vector can be used to prepare the recombinant DNA construct which can be introduced directly into the pancreatic cells.

In a preferred embodiment of the present invention, recombinant viral vectors which contain the nucleic acid encoding the IL-1β inhibitor and selectively infect the desired pancreatic β cell can be used. For example, a retroviral vector can be utilized that has been modified to delete retroviral sequences that are not necessary for packaging of the viral genome and integration into host cell DNA (see Miller A. D. et al., 1993, Meth. Enzymol. 217:581-599; Miller A. D., 1992, Curr. Top. Microbio. Immunol. 158-1-24; Guild, B. C. et al., 1988, J Virol 62: 3795-; Bramson J. et al., 1996, Hum. Gene Ther. 7: 333-342; Gao G. P. et al., 1996, J. Virol 70: 8934-8943; Wang Q. et al., 1996, Nat. Med. 2: 714-716; Wang Q. et al., 1995, Gene Ther 2: 775-783; Yeh P. et al., 1996, J. Virol 70: 559-565).

In a preferred embodiment of the invention, retroviral vectors, such as the HIV-1 lentiviral vectors which are capable of infecting non-dividing cells, may be used to transfer the nucleic acid molecule of interest into the host β cell. Such lentiviral vectors include but are not limited to those described in Zufferey et al., (1997, Nat Biotechnol 15:871-5); Naldini et al., (1996, Proc. Natl. Acad. Sci USA 93:11382-8; 1996, Science 272:263-267) and Miyoshi et al., (1997, Proc. Natl. Acad. Sci USA 94:10319-23). For a review of lentiviral vectors, see Naldini et al., (1999, In The Development of Human Gene Therapy (ed. Theodore Friedmann) Cold Spring Harbor Laboratory Press, CSH, N.Y.).

In addition, recombinant adenovirus may be used to transfer DNA into β cells. Adenoviral vectors that can be used include but are not limited to: first generation adenoviral vectors which typically have deletions in the E1 region to render them replication-defective and which may additionally have deletions in the E3 region (Gerard and Meidell, 1995, Adenovirus Vectors. In DNA cloning: A practical approach (ed. B. D. Hames and D. Glover), pp.285-306. Oxford University Press, Oxford, United Kingdom; Hitt et al., 1996, Adv. Pharmacol 40: 137-206; Spector and Samaniego, 1995, In Methods Mol. Genet 7:31-44); second generation adenoviral vectors that contain modifications in the viral E2 and E4 regions to further attenuate viral gene expression (Yeh and Perricaudet, 1997, FASEBJ. 11:615-623); and helper dependent adenovirus vectors such as those described by Hitt et al., (1999, In The Development of Human Gene Therapy (ed. Theodore Friedmann), pp.61-86, Cold Spring Harbor Laboratory Press, CSH, N.Y.) and the “gutless” viruses that maintain only the terminal repeats required for viral replication (Haecker S. E. et al., 1996, Hum Gen Ther 7: 1907; Parks, R. J. et al., 1996, Proc. Natl Acad Sci USA 93: 13565; Parks R. J. et al., 1997 J. Virol 71: 3293-3298).

Herpes simplex viral (HSV) vectors may also be used to direct the expression of Il-1β inhibitors in β cells (See, for example, Fink D. J. et al., 1995, Clin. Neuro Sci 3:284-291; Marconi P. et al., 1996, Proc. Natl Acad Sci. USA 93: 11319-11320; Fink D. J. and Glorioso J. C., 1997, Nat. Med. 3: 357-359; Fink D. J. and Glorioso J. C. 1997, Adv. Neurol 72: 149-156; Wu N. et al., 1996, J. Virol 70: 6358-6369; Zhu Z. 1996 J. Virol 70: 5346-5356; and Starr, P. A., 1996, Gene Therapy 3: 615-623. In an alternative approach, amplicons which contain an HSV origin of replication and packaging sequences can be used in conjunction with cosmids containing the HSV genome but with defective packaging sequences (Starr P. A., 1996, Gene Ther. 3:615-623; Neve R. L. and Geller A. I., 1995, Clin. Neurosci 3: 262-267; and Geller A I,1997, Adv. Neurol 72:143-148).

In accordance with the invention, adeno-associated viral vectors may be used to transfer nucleic acid molecules into a host cell, including those described in Rolling and Samulski (1995, Mol. Biotechnol 3: 9-15), Samulski (1993, Curr. Opin. Genet. Dev 3: 74-80), Miller A. D. (1997, Nat Med 3: 278-279); Fisher K. J. (1997, Nat. Med 3: 306-312); and Koeberl D. D. et al. (1997, Proc. Natl Acad. Sci USA 94: 1426-1431). For a general review of viral vectors see Robbins P. D. et al, (1998, TIBTECH 16:35) which is incorporated herein by reference.

Rather than using expression vectors which contain origins of replication, host cells can be transformed with DNA controlled by appropriate expression control elements and a selectable marker to generate β cell lines that stably express an IL-1β inhibitor. Following the introduction of the vector DNA, engineered cells may be allowed to grow 1-2 days in enriched media, and then switched to a selective media. The selectable marker in the recombinant vector confers resistance to the selection and allows cells to stably integrate the vector DNA into their chromosomes. This method may advantageously be used to engineer β cells which express inhibitors of IL-1β. Such engineered β cells may be transplanted into a host recipient to provide for the expression of inhibitors IL-1β resulting in inhibition of β cell dysfunction and apoptosis.

A number of selectable mammalian expression systems can be used to select for cells that have taken up the recombinant vector DNA, including but not limited to selection for expression of the herpes simplex virus thymidine kinase, hypoxanthine-guanine pbosphoribosyltransterase and adenine phosphoribosyl transferase protein in tk-, hgprt- or aprt-deficient cells, respectively. Also, anti-metabolic resistance can be used as the basis of selection for dihydrofolate transferase (dhfr), which confers resistance to methotrexate; xanthine-guanine phosphoribosyl transferase (gpt), which confers resistance to mycophenolic acid; neomycin (neo), which confers resistance to aminoglycoside G-418; and hygromycin B phosphotransferase (hygro) which confers resistance to hygromycin.

### 5.2. Gene Transfer

Delivery of a nucleic acid molecule encoding an IL-1β inhibitor into a host cell may be either direct, in which case the host is directly exposed to the nucleic acid or nucleic acid-carrying vector, or indirect, in which case, host cells are first transformed with the nucleic acid in vitro, then transplanted into the host. These two approaches are known, respectively, as in vivo or ex vivo gene delivery.

Recombinant vectors containing nucleic acid sequences encoding IL-1β inhibitors can be used to directly transfect target pancreatic β cells, within a host which will result in the transcription of sufficient amounts of the IL-1β inhibitor thereby alleviating the symptoms of pancreatic disorders. For example, a vector can be introduced in vivo such that it is taken up by β cells and directs the transcription of the inhibitor molecule. Such a vector can remain episomal or become chromosomally integrated, as long as it can be transcribed to produce the desired RNA. Such vectors can be constructed by recombinant DNA technology methods standard in the art as described supra.

Various delivery systems are known and can be used to directly transfer the compositions of the invention into cells, e.g. encapsulation in liposomes, microparticle bombardment (e.g., a gene gun; Biolistic, Dupont), microcapsules, receptor-mediated endocytosis (see, e.g., Wu and Wu, 1987, J. Biol. Chem. 262:4429-4432), construction of the nucleic acid as part of an appropriate nucleic acid expression vector and administering it so that it becomes intracellular, e.g., by infection using defective or attenuated viral vectors, injection of DNA, electroporation, calcium phosphate mediated transfection, etc. The compositions and methods can be used to treat pancreatic disorders in which inhibition of β cell dysfunction and apoptosis would be beneficial.

In a preferred embodiment, nucleic acids comprising a sequence encoding an inhibitor of apoptosis are administered by way of gene delivery and expression into a host cell. Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

Alternatively recombinant vectors can be used to transfect pancreatic β cells in vitro followed by transplantation of the transfected cells into the host recipient. The in vitro transfer of recombinant vectors into the β cells will result in the transcription and production of sufficient quantities of IL-1β inhibitor to prevent β cell dysfunction and apoptosis.

Ex vivo gene delivery into a cell involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art.

In an embodiment of the present invention nucleic acids encoding apoptosis inhibitors are transferred to β-host cells. Pancreatic cells may be obtained from a variety of different donor sources. In an embodiment, autologous pancreatic β-cells are obtained from the subject who is to receive the transplanted cells. This approach is especially advantageous since the immunological rejection of foreign tissue and/or a graft versus host response is avoided. In yet another preferred embodiment of the invention, allogenic pancreatic cells may be obtained from donors who are genetically related to the recipient and share the same transplantation antigens on the surface of their pancreatic cells. Alternatively, if a related donor is unavailable, pancreatic cells from antigenically matched (identified through a national registry) donors may be used.

Pancreatic cells can be obtained from the donor by standard techniques known in the art. In an embodiment of the invention, pancreatic cells are isolated from disaggregated pancreatic tissue biopsy. This may be readily accomplished using techniques known to those skilled in the art. For example, the pancreatic tissue can be disaggregated mechanically and/or treated with digestive enzymes and/or chelating agents that weaken the connections between neighboring cells, making it possible to disperse the tissue suspension of individual cells. Enzymatic dissociation can be carried out by mincing the pancreatic tissue and treating the minced tissue with any of a number of digestive enzymes. Such enzymes include, but are not limited to, trypsin, chymotrpsin, collagenase, elastase and/or hylauronidase or liberase-HI. A review of tissue disaggregation technique is provided in, e.g., Freshney, Culture of Animal Cells, A Manual of Basic Technique, 2d Ed., A. R. Liss, Inc., New York, 1987, Ch. 9).

In addition, the engineered pancreatic cells may be attached in vitro to a natural or synthetic matrix that provides support for the transplanted cells prior to transplantation. The type of matrix that may be used in the practice of the invention is virtually limitlessness. The matrix will have all the features commonly associated with being “biocompatible”, in that it is in a form that does not produce an adverse, or allergic reaction when administered to the recipient host. Growth factors capable of stimulating the growth of the pancreatic cells may also be incorporated into the matrices. Such matrices may be formed from both natural or synthetic materials and may be designed to allow for sustained release of growth factors over prolonged periods of time. Thus, appropriate matrices will both provide growth factors and also act as an in situ scaffolding in which the transplanted cells can proliferate. In preferred embodiments, it is contemplated that a biodegradable matrix that is capable of being reabsorbed into the body will likely be most useful. To improve pancreatic cell adhesion to the matrix, and survival and function of the pancreatic cell, the matrix may optionally be coated on its external surface with factors known in the art to promote cell adhesion, growth or survival. Such factors include cell adhesion molecules, extra cellular matrix molecules and/or growth factors.

In yet another embodiment of the invention, cell types other than β cells, may be genetically engineered to express inhibitors of IL-1β using the methods and nucleic acid molecules described supra. In particular, cell types other than β cells may be utilized when the inhibitor of IL-1β is a secreted polypeptide. Such engineered cells may then be transplanted into the host recipient to provide β cell protection from IL-1β mediated apoptosis.

### 5.3. Effective Doses and Methods of Administration

When in vivo methods of gene delivery are utilized, the nucleic acids encoding the apoptosis inhibitors will be administered in amounts which are effective to produce the desired effect in the targeted cell, i.e., inhibition a β cell dysfunction and apoptosis. Effective dosages of the nucleic acids can be determined through procedures well known to those in the art which address such parameters as biological half-life, bio availability and toxicity. The amount of the composition of the invention which will be effective will depend on the nature of the pancreatic disease or disorder being treated, and can be determined by standard clinical techniques.

Determination of effective amounts is well within the capability of those skilled in the art. The effective dose may be determined by using a variety of different assays designed to detect restoration of pancreatic function. The progress of the transplant recipient can be determined using assays including assays to detect blood sugar or insulin levels. In addition, in vitro assays may optionally be employed to help identify optimal dosage ranges for prevention of nitric oxide production and/or inhibition of apoptosis.

Ex vivo methods of gene delivery will employ β cells genetically engineered as described above to enable them to produce functionally active biologically proteins capable of inhibiting apoptosis. The transfected β cells will be administered to the recipient in an effective amount to achieve its intended purpose. More specifically, an effective amount means an amount sufficient to produce proteins capable of inhibiting β cell dysfunction and apoptosis thereby alleviating the symptoms associated with pancreatic disorders. The number of cells needed to achieve the purposes of the present invention will vary depending on the degree of pancreatic damage and the size, age and weight of the host. For example, the cells are administered in an amount effective to restore pancreatic function. The dose range of cells to be used in the practice of the invention may vary between 10⁷-10⁴ cells, although the preferable dose of administered cells will be between 10⁶-10⁵. It may be necessary to use dosages outside these ranges in some cases, as will be apparent to those of skill in the art.

The pancreatic cells can be administered to the recipient in one or more physiologically acceptable carriers. Carriers for these cells may include, but are not limited to, solutions of phosphate buffered saline (PBS) containing a mixture of salts in physiologic concentrations. In addition, the β cells may be associated with a matrix prior to administration into the recipient host.

The methods of the present invention encompass administration of the transfected β cells into the recipient so as to become located in the pancreas. The administration of the β cells, is accomplished by conventional techniques such as surgical transplantation of the transfected cells into the recipient host pancreas. In some instances it may be necessary to administer the pancreatic cells more than once to restore β cell function.

In another embodiment of the invention, the transfected β cells can be administered by intravenous infusion. The cells to be injected, are drawn up into a syringe and injected into the recipient host. In such instances, the cells would be expected to migrate to the recipient's liver where they will function to provide the recipient with the required β cell functions, including for example, sufficient levels of secreted insulin.

Immunosuppressive reagents may also be co-administered to the recipient host to prevent graft rejection resulting from the transplant recipient's immune response against donor major histocompatibility antigens expressed on the surface of the donor β cells. Such reagents include but are not limited to cyclosporine, corticosteroids, azathioprine, tacrolimus and mycophenolate mofetil. Determination of effective amounts of such immunosuppressive reagents is well within the capability of those skilled in the art.

In addition, β cells may be engineered prior to transplantation to express immunosuppressive molecules in addition to IL-1β inhibitors to prevent graft rejection. Molecules capable of suppressing an immune response and improving graft survival, include for example, cytokines such as transforming growth factor β1, viral encoded IL-10 and IL-4. Alternatively, non-β cell types may be genetically engineered to express immunosuppressive molecules and can be co-transplanted with the donor β cells to prevent graft rejection.

The present invention provides for compositions comprising an effective amount of a nucleic acid encoding a protein capable of inhibiting β cell dysfunction and apoptosis, or a host cell genetically engineered to express an inhibitor of apoptosis, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical sciences” by E. W. Martin.

The present invention also provides a pharmaceutical pack or kit comprising one or more containers filled with one or more unit dosage forms containing the active ingredient of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLE

### Adenoviral Gene Transfer of the Interleukin-1 Receptor Antagonist Protein to Human Islets

The following subsection demonstrates that adenoviral gene therapy of the cDNA encoding the interleukin-1 receptor antagonist protein (IL-IRa) to cultured islets results in protection of human islets against IL-1B-induced nitric acid formation, impairment in glucose-stimulated insulin production and Fas-triggered apoptosis activation.

### 6.1. Materials and Methods

### 6.1.1. Generation of Recombinant Adenoviruses

E1 and E3 deleted adenoviral vector encoding enhanced green fluorescent protein (Ad-eGFP) was constructed as described (Hardy S. et al., 1997, J Virol 71: 1842; Mittereder N. et al, 1996, J Virol 70: 7498) by Cre-lox recombination with reagents generously provided by C. Hardy (Somatix Inc; Alameda, Calif.). Briefly, a SnaBI-HpaI fragment containing part of the cytomegalovirus promoter, the eGFP cDNA and part of the SV40 poly(A) sequence, was inserted into the pAdlox shuttle plasmid. E1-substituted recombinant adenovirus was generated by cotransfection of SfiI-digested pAdlox-transgene and ψ5 helper virus DNA into the adenoviral packaging cell line CRE8. The virus was propagated and purified as described (Hardy S. et al., 1997 J. Virol 71: 1842). The E1 and E3 deleted Ad-LacZ was provided by I. Kovesdi (Genvec, Rockville, Md.) whereas the Ad-IL-1Ra virus was provided by Amgen, Inc. (Boulder, Colo.). The transgene is driven by the CMV promoter in both the Ad-LacZ and Ad-IL-1Ra vectors.

### 6.1.2. Isolation of Human Islets

Pancreata from multiorgan cadaver donors were provided by the NDRI (National Disease Research Interchange, Philadelphia, Pa.) and local OPOs (Organ Procurement Organizations) with the appropriate consent for research use. Human islets were obtained using the automated method and Liberase-HI enzyme blend (Roche/Boehringer-Mannheim, Indianapolis, Ind.) for tissue dissociation (Linetsky E. et al., 1997, Diabetes 46: 1120). The islets were purified by centrifugation on discontinuous density gradients using a COBE-2991 Cell Processor (Cobe, Lakewood, Colo.) (Linetsky E. et al., 1997, Diabetes 46: 1120). Islets with a purity greater than 80% were routinely obtained.

### 6.1.3. Gene Transfer of β-galactosidase,eGFP and Human IL-1Ra to Islets in Culture Using E1-E3-deleted Adenoviral Vectors

Islets were washed twice in serum free RPMI 1640 (Gibco-BRL) supplemented with a 1% penicillin/streptomycin solution (Gibco-BRL) prior to infection. 200-300 islets were infected with Ad-LacZ, Ad-eGFP or Ad-IL-1Ra at a pfu (plaque forming unit) of 1×10⁶ in a minimal volume of serum-free RPMI 1640 for 2-4 hours at 37° C. Following the infection, the islets were washed twice in serum-free medium and then once with medium containing 10% heat-inactivated fetal calf serum (Gibco-BRL). Islets were then incubated at 37° C. in medium with serum for two days, after which time all the assays were carried out. All the functional assays described below were performed in triplicate on at least three different occasions unless otherwise indicated.

### 6.1.4. Detection of Secreted Transgene Products and Evaluation of β Cell Function Following IL-1β Treatment

β-galactosidase was detected by X-gal staining and eGFP fluorescence was visualized microscopically under standard excitation/emission parameters. Secreted IL-1Ra protein was detected in the culture supernatants two days following infection using commercially-available ELISA kits (R&D Systems). This also coincided with the day of IL-1β treatment in the designated cultures. To assess the effects of IL-1β on β cell function of genetically-modified and unmodified islets, glucose-stimulated insulin secretion was used as a functional assay. Islets were first treated with 50 U of recombinant human IL-1β (Sigma) for a period between 18-24 hours immediately following a preincubation in fresh media for 16-24 hours. The IL-1β-containing medium was removed and the islets were washed twice with Krebs-Ringer-HEPES buffer (KRH buffer; 25 mM HEPES pH 7.4, 115 mM NaCl, 24 mM NaHCO₃, 5 mM KCl, 2.5 mM CaCl₂, 1 mM MgCl₂). Incubation was carried out at 37° C. in KRH buffer for 30 minutes followed by an additional incubation for 30 minutes in the presence of 5 or 18 mM glucose (final concentration). The buffer was subsequently removed and its insulin content was determined by a commercially-available ELISA kit (Dako Chemicals) which specifically recognizes processed insulin.

In order to evaluate NO production, the islet culture supernatants were collected between 18-24 hours following the addition of IL-1β and an aliquot was subjected to the Griess reaction.

### 6.1.5. Assessment of Apoptosis Activation in Vitro

Uninfected islets, as well as those infected with Ad-eGFP, or Ad-IL-1Ra, were treated with 50 units IL-1β for 24 hours as described above. Furthermore, a subset of islets (pretreated or not with IL-1β) were challenged with the agonistic human Fas antibody (clone CH-11, Upstate Biotechnology Inc.) for 1 hour at 37° C. At the end of the incubation, islets were lysed and processed for the detection of Caspase-3 (CPP32) activity using a commercially available kit (ApoAlert, Clontech, Calif.). As an indirect means of correcting for cell number, the CPP32 activity was corrected by the number of nanograms of DNA in the lysate assayed, using the PicoGreen reagent, an intercalating DNA fluorogenic compound (Molecular Probes Inc., Oreg.).

### 6.1.6. Statistics

Statistics were performed using the SPSS for Windows v. 8.0 package and differences among experimental groups at a p level less than 0.05 using the two-tailed Student's t-test were considered statistically significant.

### 6.2. Results

### 6.2.1. IL-1Ra is Produced at High Levels From Genetically-modified Human Islets in Vitro

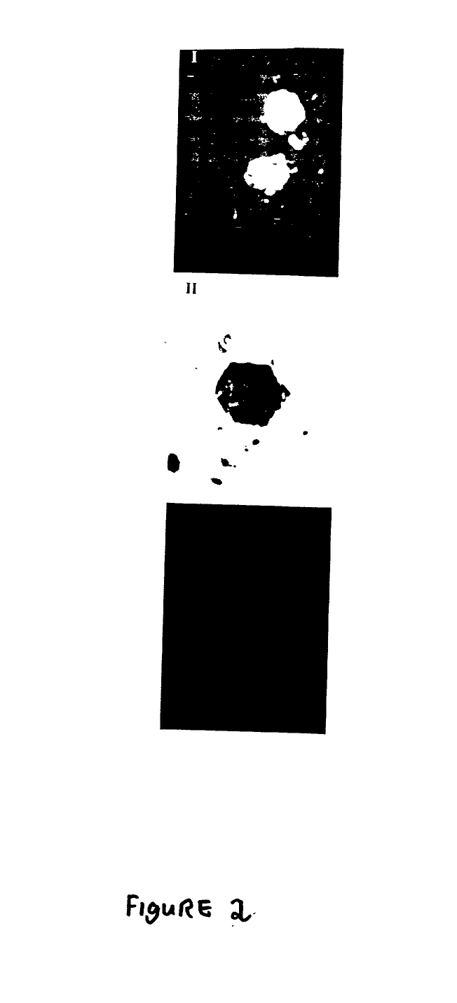

Recent results from a number of laboratories have demonstrated that adenoviruses are very efficient at infecting human and rodent islets in vitro (Weber M. et al, 1997, J. Surg Res 69: 23). The ability to infect human islets with adenoviral vectors at low multiplicities of infection (MOI) was tested. 200-300 human islets are routinely cultured for no more than 10 days following their removal from the cadaveric donor. Within this period the islets were infected in serum-free medium. Shown in FIG. 2 are representative results of human islets infected with 1×10⁶ pfu of Ad-eGFP (Panel I) and Ad-LacZ (Panel II). Panel III shows mock-infected islets (islets not exposed to virus, but to vehicle only) under fluorescence microscopy. Approximately 70% of the cells within the islet were infected with the eGFP virus as determined by dispersing the cells followed by FACS analysis. The secretion of IL-1Ra into the culture supernatant using commercially-available ELISA kits was also examined. Table I shows that IL-1Ra was secreted to levels as high as 31 ng/48 hours with no detectable IL-1Ra in the media of uninfected islets or in those of islets infected with Ad-LacZ or Ad-eGFP. These results demonstrate that human islets can be infected efficiently with adenoviral vectors and that they can secrete significant levels of IL-1Ra.

TABLE I
IL-1Ra production (ng/mL)

| Control | Ad-LacZ/Ad-eGFP | Ad-IL-1RA |
|---|---|---|
| undetectable | undetectable | 31 +/− 0.76 |

### 6.2.2. Adenoviral Gene Transfer of IL-1Ra Leads to Protection from IL-1β-induced Impairment of Insulin Secretion Following a Glucose Challenge

The ability of genetic modification of islets by Ad-IL-1Ra to inhibit or suppress the IL-1β-induced impairment of insulin secretion in vitro in response to a glucose stimulation was examined. In parallel, the IL-1β-induced production of NO by infected and uninfected islets was also examined. The percent changes in insulin secretion are shown in FIG. 2 with the actual insulin secretion profile for each treatment group shown in Table 2.

TABLE 2
Glucose-stimulated insulin release assay

| | Insulin release % +/− SEM | Insulin release % +/− SEM |
|---|---|---|
| Islet Status | (5 mM glucose) | (18 mM glucose) |
| Control | 100 | 866 +/− 200 |
| Ad-LacZ | 113 +/− 80 | 900 +/− 133 |
| IL-1β | 66 +/− 53 | 80 +/− 13 |
| Ad-LacZ + | 93 +/− 6 | 80 +/− 26 |
| IL-1β | | |
| Ad-IL-1Ra | 364 +/− 250 | 1029 +/− 400 |
| Ad-IL-1Ra + | 333 +/− 133 | 733 +/− 166 |
| IL-1β | | |

The production of insulin by untreated, uninfected islets in the presence of 5 mM glucose is taken as control (100%). In uninfected islets, insulin secretion in response to high glucose concentration (18 mM final) was impaired in islets exposed to IL-1β. In contrast, the islets that were infected with adenovirus encoding IL-1Ra maintained a normal insulin secretory response to glucose following IL-1β treatment. The response was almost identical to unmodified islets that were not treated with IL-1β, although the amount of insulin secreted in the presence of 5 mM glucose in Ad-IL-1Ra-infected islets was greater than that from uninfected islets exposed to the same concentration of glucose. The adenovirus itself does not affect islet response to high glucose levels in the absence of IL-1β as demonstrated using an adenovirus encoding the β galactosidase transgene (Ad-LacZ). Furthermore, Ad-LacZ-infected islets respond to IL-1β in an identical manner as do the mock-infected islets.

### 6.2.3. IL-1Ra Expression Suppresses No Production

The ability of genetic modification of islets by Ad-IL-1Ra to inhibit or suppress the IL-1β-induced NO production was next examined. Total NO production in the culture supernatants of IL-1β-treated unmodified and modified islets was determined using the Griess reaction to measure nitrite in the culture supernatant. Seventy two hours following the infection with Ad-IL-1Ra or Ad-LacZ, the media of the cultures were replaced with fresh media and then, after an additional 16-24 hours, 50 units of IL-1β were added for a further 18-24 hour incubation. Normal islets and those infected with Ad-LacZ produced detectable levels of NO in the absence of any IL-1β in all experiments. This level increased significantly following IL-1β treatment. In cultures that were infected with Ad-IL-1Ra, NO production was not statistically different from that detected in islets not subjected to IL-1β. Ad-LacZ-infected islets exposed to IL-1β produced the most NO whereas exposure of Ad-IL-1Ra-infected islets to IL-1β had no effect on stimulating NO production relative to mock-infected islets. Finally, islets infected with Ad-IL-1Ra appeared to have a reduction in the amount of basal NO produced.

### 6.2.4. Adenoviral Delivery of IL-1Ra Suppresses IL-1β-induced, FAS-triggered Apoptosis Activation of Islets In Vitro

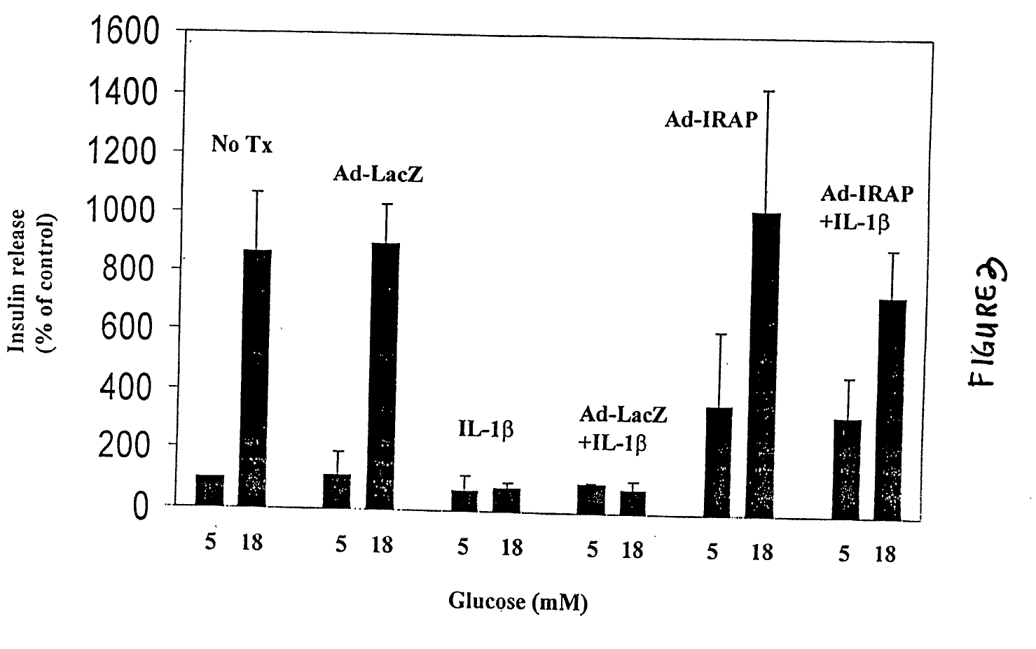

Fas expression at the cell surface of islets is increased following IL-1β treatment, but recent reports indicate that IL-1β itself may also induce apoptosis in a Fas-independent manner. To determine whether islet-derived IL-1Ra could prevent IL-1β-induced, Fas-mediated apoptosis activation following adenoviral gene transfer, Capase 3 activity was used a marker. Caspase-3 is specifically stimulated following ligation of the Fas antigen and its activity is therefore a reliable marker of apoptosis induction (Fernandes-Alnemri, T. et al., 1994, J. Biol Chem 269: 30761; Casciola-Rosen L. 1996, J. Exp. Med. 183: 1957; Lazebnik Y. A. et al., 1994, Nature 371: 346). Caspase-3 activity was examined one hour after treating islet cultures with the agonistic Fas antibody (CH-11) with activity corrected for cell number by determining the total amount of DNA in the cell lysate using PicoGreen. As shown in FIG. 3, CH-11 treatment of control or Ad-eGFP-infected islets following IL-1β treatment induces significant caspase-3 activity relative to untreated islets. In contrast, infection with Ad-IL-1Ra completely protected the islets from caspase-3 activation following IL-1β and Fas antibody treatment. The level of caspase-3 activity in untreated or CH-11-treated control islets also was significantly reduced by infection with Ad-IL-1Ra. These results demonstrate that expression of IL-1Ra by islets is able to suppress Fas-mediated apoptosis following IL-1β treatment.

### 7. EXAMPLE

### Adenoviral Gene Transfer of Human Insulin-like Growth Factor-I Gene to Human Islets

The following subsection describes data demonstrating that adenoviral gene transfer of the human insulin-like growth factor-I (IGF-1) cDNA can prevent IL-1B-mediated nitric oxide production from human islets in vivo as well as suppression of β cell function as determined by glucose-stimulated insulin production. Moreover, IGF-1 gene transfer can prevent IL-1B induced, Fas-triggered caspase-3 activity, a marker of apoptosis activation.

### 7.1. Materials and Methods

### 7.1.1 Generation of Recombinant Adenoviruses

E1-deleted adenoviruses encoding β galactosidase (Ad-LacZ), enhanced green fluorescent protein (Ad-eGFP), and IGF-I (Ad-IGF-I), were designed and titered as described (Hardy S. et al., 1997, J Vinol 71: 1842-9; Mittereder N. et al., 1996, J Virol 70: 7498-509) by Cre-lox recombination with reagents generously provided by Somatix Inc. (Alameda, Calif.). Briefly, a SnaBI-HpaI fragment containing part of the cytomegalovirus promoter, the transgene CDNA (β galactosidase, eGFP or IGF-I) and part of the SV40 poly(A) sequence, was inserted into the pAdlox shuttle plasmid. E1-substituted recombinant adenovirus was generated by cotransfection of SfiI-digested pAdlox-transgene and ψ5 helper virus DNA into the adenoviral packaging cell line CRE8, propagated and purified as described (Hardy S. et al., 1997, J Virol 71: 1842-9; Mittereder N. et al., 1996, J Virol 70: 7498-509).

### 7.1.2. Isolation and Culture of Human Islets

Human islets were obtained using the Liberase enzyme blend. Pancreata were obtained from cadaveric donors and subjected to the digestion, isolation and purification as described (Linetsky E. et al., 1997, Diabetes 46: 1120-3). Islets were routinely obtained to a purity greater than 80% (mantled islets).

### 7.1.3. Gene Transfer of β-galactosidase, eGFP and Human IGF-I to Islets in Culture Using E1-E3-deleted Adenovirus Vectors

Islets were washed twice in serum free RPMI 1640 (Gibco-BRL) supplemented with a 1% penicillin/streptomycin solution (Gibco-BRL) prior to infection. 200-300 islets were infected with Ad-LacZ, Ad-eGFP or Ad-IGF-I at a pfu (plaque forming units) of 1×10⁶ in a minimal volume of serum-free RPMI 1640 for 2-4 hours at 37° C. Following the infection, the islets were washed twice in serum-free medium and then once with medium containing 10% heat-inactivated fetal calf serum (Gibco-BRL). Islets were then incubated at 37° C. in medium with serum for two days after which time all the assays were carried out. All the functional assays described below were performed in triplicate on at least three different occasions unless otherwise indicated.

### 7.1.4. Detection of Secreted Transgene Products and Evaluation of B Cell Function Following IL-1β Treatment

β-galactosidase was detected by X-gal staining and eGFP fluorescence was visualized microscopically under standard excitation/emission parameters. Secreted IGF-I protein was detected in the culture supernatants two days following infection using commercially-available ELISA kits (IDL Laboratories). This also coincided with the day of IL-1β treatment in the designated cultures. To assess the effects of IL-1β on β cell function of genetically-modified and uiimodified islets, glucose-stimulated insulin secretion was used as a functional assay. Islets were first treated with 50 U of recombinant human IL-1β (Sigma) for a period between 18-24 hours immediately following a preincubation in fresh media between 16-24 hours. The IL-1β-containing medium was removed and the islets were washed twice with Krebs-Ringer-HEPES buffer (KRH buffer; 25 mM HEPES pH 7.4, 115 mM NaCl, 24 mM NaHCO₃, 5 mM KCl, 2.5 mM CaCl₂, 1 mM MgCl₂). Incubation was carried out at 37° C. in KRH buffer for 30 minutes followed by an additional incubation for 30 minutes in the presence of 5 and 18 mM glucose (final concentration). The buffer was subsequently removed and its insulin content was determined by a commercially-available ELISA kit (Dako Chemicals) which specifically recognizes processed insulin.

In order to evaluate NO production, the islet culture supernatants were collected between 18-24 hours following the addition of IL-1β and an aliquot was subjected to the Griess reaction.

### 7.1.5. Assessment of Apoptosis Activation in vitro

Uninfected islets, as well as those infected with Ad-LacZ or Ad-IGF-I were treated with 50 units IL-1β for 24 hours as described above. Furthermore, a subset of islets (pretreated or not with IL-1β) were challenged with the agonistic human Fas antibody (clone CH-11, Upstate Biotechnology Inc.) for 1 hour at 37° C. At the end of the incubation, islets were lysed and processed for the detection of caspase-3 (CPP32) activity using a commercially available kit (ApoAlert, Clontech, Calif.). As an indirect means of correcting for cell number, the CPP32 activity was corrected by the number of nanograms of DNA in the lysate assayed, using the PicoGreen reagent, an intercalating DNA fluorogenic compound (Molecular Probes Inc., Oreg.).

### 7.1.6. Statistics

Statistics were performed using the SPSS for Windows v. 8.0 package and a p value less than 0.05, using a two-tailed Student's t-test, was taken to indicate statistically significant differences.

### 7.2.1. AD-IGF-1 Gene Transfer to Islets Results in High IGF-1 Protein Secretion

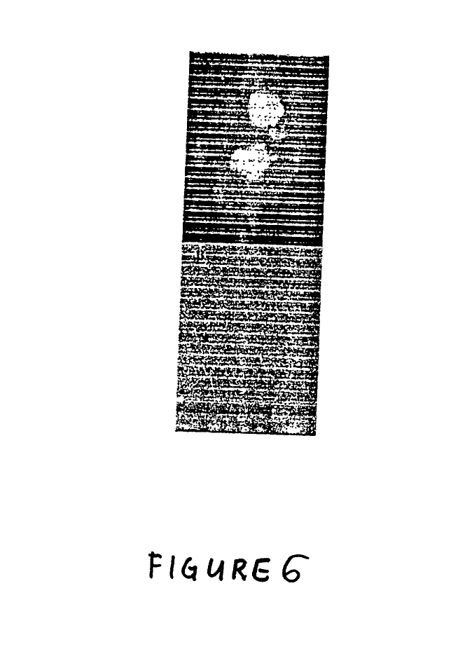

Adenoviral gene transfer to human and murine islets is very efficient (Csete M. E. et al., 1994 Transplant Proc 26: 756; Csete M. E. et al., 1995, Transplantation 59: 263-8; Wiber M. et al., 1997 J. Surg Res 69: 23-32), and infection of human islets with recombinant, replication-defective adenoviruses deleted for E1 and E3, can result in very high levels of transgene expression. Groups of 200-300 islets isolated were cultured no later than 48-72 hours following islet isolation from cadaveric donor pancreas. Islets were infected with Ad-IGF-I as well as Ad-eGFP and Ad-LacZ at identical pfu (1×10⁶) in a minimal volume of serum-free medium. FIG. 6 demonstrates efficient eGFP reporter gene expression in human islets following adenoviral gene transfer. IGF-I protein was detected in the culture supernatant using a commercially-available ELISA kit in which IGF-I was measured following acid-ethanol extraction to remove IGF binding proteins.

IGF-I in the media of uninfected islets was detected at significant levels (28.4+/−2.6 ng/mL) and in Ad-IGF-I-infected islets, we detected IGF-I at 102.8+/−34.2 ng/mL (Table 3).

TABLE 3
Human IGF-I levels (ng/mL)

| Control | Ad-IGF-I |
|---|---|
| 28.4 +/− 2.6 | 102.8 +/− 34.2 |

### 7.2.2. Prevention of IL-1-β-induced Suppression of Glucose-simulated Insulin Release as Well as Nitric Oxide Production with Adenoviral Gene Transfer of IGF-I to Human Islets In Vitro

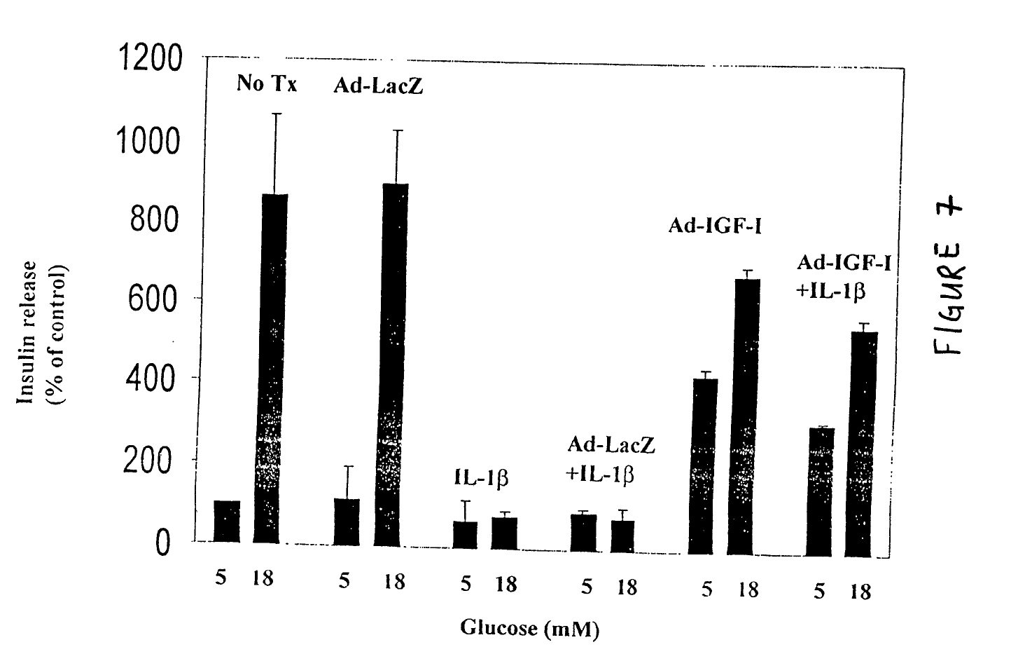

FIG. 7 shows that 50 units of IL-1β is sufficient to impair the ability of islets to respond to a high glucose concentration (18 mM), both in uninfected islets as well as in those infected with a control virus (Ad-LacZ). Islets infected with Ad-IGF-I secreted higher insulin relative to uninfected and Ad-LacZ-infected islets when exposed to glucose at 5 mM final concentration. The difference was statistically significant (430+/−18.4% from Ad-IGF-I-infected islets vs. 113+/−80% in Ad-LacZ-infected islets where uninfected islets exposed to 5 mM glucose are taken to represent 100% as control, p<0.05). As shown in Table IV, Ad-IGF-I-infected islets were refractory to the effects of IL-1β in the presence of high glucose and secreted significantly greater insulin at 18 mM glucose than islets exposed to 5 mM. The difference was statistically significant (p<0.05).

TABLE 4
Glucose-stimulated insulin release assay

| | | |
|---|---|---|
| Control | 100 | 866 +/− 200 |
| Ad-LacZ | 113 +/− 80 | 900 +/− 133 |
| IL-1β | 66 +/− 53 | 80 +/− 13 |
| Ad-LacZ + IL-1β | 93 +/− 6 | 80 +/− 26 |
| Ad-IGF-I | 430 +/− 18.4 | 676 +/− 22.5 |
| Ad-IGF-I + IL-1β | 316 +/− 4.1 | 553 +/− 20.5 |

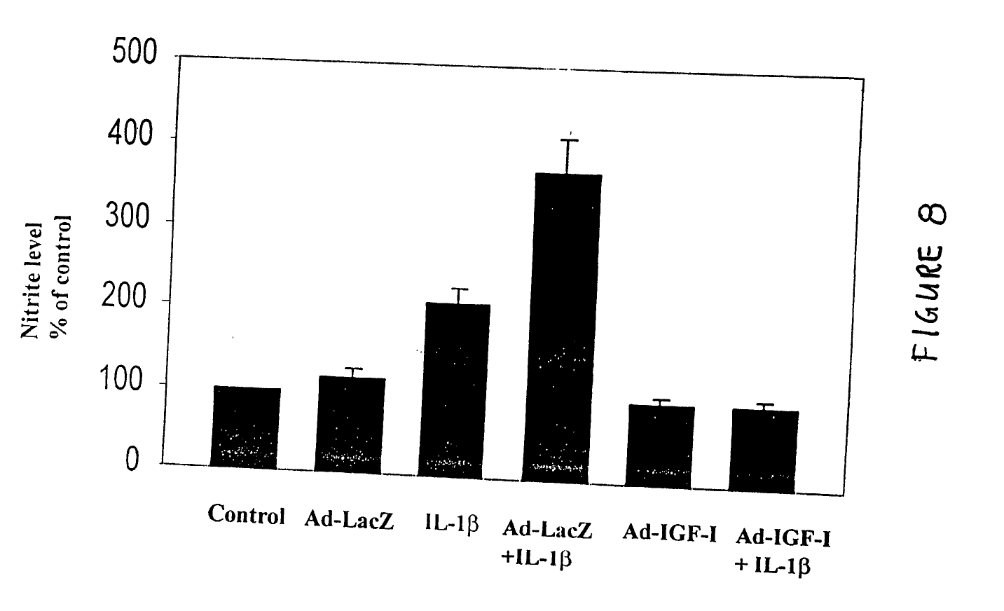

As nitric oxide has been shown to be an important determinant of the suppression of IL-1β-induced impairment of glucose-stimulated insulin secretion, the level of nitrite in the culture supernatant of mock-infected, Ad-LacZ- and Ad-IGF-I-infected islets in the presence or absence of 50 units of IL-1β using the Griess reagent was evaluated. Mock-infected islets as well as those infected with Ad-LacZ produced a basal amount of nitric oxide in the absence of any IL-1β. This observation was made in every single experiment (n=3, in triplicate). There was a significant increase of NO production when islets were exposed to 50 units of IL-1β (213+/−15.3% relative to control, p<0.05). Ad-IGF-I-infected islets did not produce NO at significantly higher levels than mock-infected or Ad-LacZ-infected islets not exposed to IL-1β (Ad-IGF-I: 100+/−5%, Ad-LacZ: 116+/−10% relative to uninfected control islets) (FIG. 8). In the presence of IL-1β, however, Ad-LacZ-infected islets displayed a large increase in NO production relative to uninfected islets exposed to IL-1β (375+/−40% vs. 213+/−15.3% IL-1β-treated uninfected islets, p<0.05). Finally, Ad-IGF-I-infected islets exposed to IL-1β produced NO at levels no higher than mock-infected islets unexposed to IL-1β (Ad-IGF-I: 100+/−5.8% vs. control; IL-1β-treated islets : 213% +/−15.3 vs. control, p<0.05 Ad-IGF-I vs. uninfected islets in the presence of IL-1β).

### 7.2.3. Human Islets Infected with AD-IGF-I are Protected From IL-1β Stimulated, Fas-triggered Apoptosis Activation in Vitro

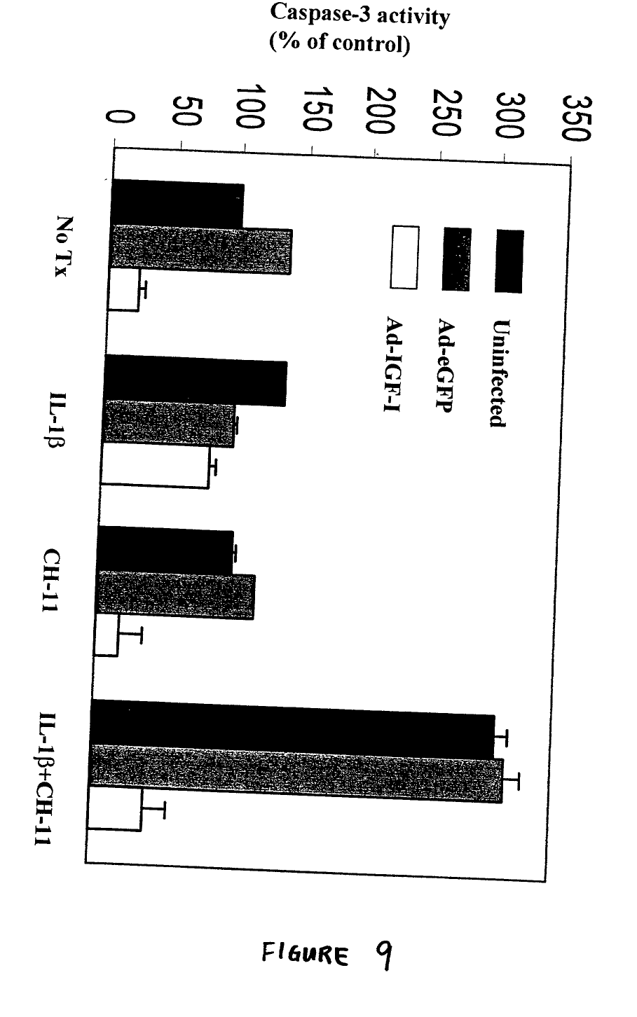

An agonistic Fas antibody was used to induce apoptosis in untreated mock-infected and infected islets as well as those exposed to IL-1β. Caspase-3 activity was assessed as a marker of apoptosis induction since recent reports demonstrate that it is specifically activated in response to the agonistic Fas antibody used same antibody used here. In this set of experiments, the Ad-eGFP was used as a control virus. In previous experiments it was shown that Ad-eGFP infection of human islets in vitro does not affect their function. IL-1β was added to mock-infected, Ad-eGFP and Ad-IGF-I-infected islets and the agonistic antibody was then added for 1 hour. Caspase-3 activity was normalized to the number of cells in the lysate by an indirect quantitative method whereby the amount of DNA is evaluated using a fluorescent intercalating agent. In FIG. 9, caspase-3 activity was significantly suppressed in islets infected with Ad-IGF-I compared to uninfected islets (23+/−5% relative to control, where 100% represents the caspase-3 activity in mock-infected, untreated islets, p<0.05). There were no significant differences in caspase-3 activity among untreated, uninfected islets and those exposed to IL-1β or the agonistic antibody CH-11. Pretreatment of uninfected islets with IL-1β followed by exposure to CH-11 resulted in a significant increase in caspase-3 activity (307+/−10.7% in IL-1β+CH-11-treated islets vs. 137+/−1.3% in IL-1β-treated islets alone and 101+/−3.7% in islets treated only with CH-11, p<0.05 for IL-1β+CH-11 vs. control). The reduction in caspase-3 activity in Ad-IGF-I-infected islets compared to control, was abolished in Ad-IGF-I-infected islets exposed to IL-1β, however, the level was almost identical to that in control islets (FIG. 9). The most striking observation is the suppression of IL-1β-stimulated, Fas-triggered caspase-3 activation in islets infected with Ad-IGF-I. Compared to uninfected islets and those infected with Ad-eGFP that were exposed to IL-1β and the agonistic CH-11 antibody, IGF-I-expressing islets exhibited statistically-significant lower levels of caspase-3 (40+/−20% compared to 307+/−10.7% in control islets or 313+/−14% in Ad-eGFP infected islets; p<0.05 in both cases).

### 8. EXAMPLE

### Infection of Human Islets by a Lentiviral Vector

The following subsection describes experiments demonstrating successful gene therapy into human islet cells using a lentiviral vector. The lentiviral vector was able to express significant levels of the interleukin-1 receptor antagonist protein following infection. Moreover, there was no impairment of islet β cell function following infection as demonstrated by a normal response in glucose stimulation.

### 8.1. Materials and Methods

### 8.1.1. Vector Preparation

An E1-deleted adenovirus encoding β-galactosidase (Ad-LacZ) or enhanced green fluorescent protein (Ad-eGFP) was designed and titered as described Hardy S. et al., (1997, J Virol 71: 1842); Mittereder N. et al, (1996, J. Virol 70: 7498) by Cre-lox recombination with reagents generously provided by Somatix Inc. (Alameda, Calif.). For the construction of the Ad-eGFP vector, a SnaBI-HpaI fragment containing part of the cytomegalovirus promoter, the enhanced green fluorescent protein (eGFP) cDNA and part of the SV40 poly(A) sequence (derived from PEGFP; Clontech) was inserted into the pAdlox shuttle plasmid. E1-substituted recombinant adenovirus was generated by co-transfection of SfiI-digested pAdlox-eGFP and ≠5 helper virus DNA into the adenoviral packaging cell line CRE8, propagated and purified as described by Hardy et al., (1997, J. Virol 71: 1842). Ad-IL-iRa virus was obtained from Amgen (Boulder, Colo.) and Ad-LacZ was generously provided by Genvec Inc. (Bethesda, Md.). Subsequent propagation was performed as described by Hardy S. (1997, J. Virol 71: 1842-9) and Mittlereder N. et al., (1996, J. Virol 70: 7498).

The vesicular stomatitis virus G-protein (VSV-G)-pseudotyped lentiviral vectors encoding either β galactosidase (LtV-LacZ), enhanced green fluorescent protein (LtV-eGFP) or the human IL-1Ra cDNA (LtV-IRAP) were generated using a three-plasmid system (Zufferey R. et al., 1997, Nat Biotechnol 15:871; and Naldini L. et al., 1996, Proc Natl Acad Sci USA 93: 11382). Briefly, Subconfluent 293T cells were co-transfected with the three plasmids and incubated for two days in DMEM medium with 10% FCS, supplemented with 1% penicillin/streptomycin (Gibco-BRL) and containing L-glutamine. Following this incubation, the media were replaced with fresh media and after an overnight incubation, the media was collected, filtered through a 0.22 micron filter and ultracentrifuged at 25 000 rpm in an SW-41Ti rotor (Beckman) for 1 hr. 30 min. at 4° C. The resulting viral pellet was dissolved in a minimal volume of Hank's balanced salt solution overnight and LtV-LacZ, LtV-eGFP were then titered on 293T cells.

### 8.1.2. Infection of Human Islets in Vitro

Human islets were obtained using the Liberase enzyme blend. Pancreata were obtained from cadaveric donors and subjected to the digestion, isolation and purification (Linetsky E. et al., 1997 Diabetes 46: 1120). Islets were routinely obtained to a purity greater than 80% (mantled islets).

Groups of 200 islets were infected with Ad-LacZ or Ad-eGFP (1-2×10⁶ pfu) for 4 hours in a minimal volume of serum-free RPMI 1640 medium (Gibco-BRL) at 37° C. The islets were then washed extensively with serum-free RPMI 1640 and incubated for an additional 48 hours in RPMI 1640 with 10% FCS. For LtV-LacZ or LtV-eGFP infection, an equal number of islets was cultured overnight in a minimal volume of concentrated viral supernatant adjusted to contain 1×10⁶ TIU and 8 μg/mL polybrene. The islets were then washed extensively with RPMI 1640 and the incubation was continued for an additional 48 hours in RPMI 1640 with 10% fetal calf serum. For LtV-IL-1Ra, an equivalent number of islets were incubated in a minimal volume of medium containing 100 μL of concentrated viral supernatant. At the end of the incubation, cultures were processed either for in situ β galactosidase expression (Ad-LacZ and LtV-LacZ), dispersed into single cells for FACS analysis (Ad-eGFP and LtV-eGFP) or the media assayed for IL-1Ra (LtV-IL-1Ra) by ELISA. For β galactosidase, the islets were washed twice with sterile PBS and then fixed in 0.5% glutaraldehyde/PBS for 30 minutes-1 hour at 4° C. The glutaraldehyde was removed with three washes of PBS/1 mM MgCl₂ and stained with X-gal for 2-4 hours at 37° C.

For FACS analysis, intact islets were dispersed into single cell suspensions (Peakman M. et al., 1994 Transplantation 57: 384-93). The cell suspensions were subjected to FACS analysis in a Becton-Dickinson FACStar Plus, with an argon laser set at 488 nm and with gated cells having a forward scatter between 1-15°. Under this selection, the majority of cells gated are β cells (Nielson, D. A. et al., 1982, Diabetes 31: 299-306).

IL-1Ra was detected by ELISA (R&D Systems) 4 days later in the media as recommended by the manufacturer.

### 8.1.3. Functional Evaluation of Modified Intact Islets by Glucose-stimulated Insulin Production

To determine if viral infection affected β cell function, the response of islets to a glucose challenge was tested by measuring insulin release into serum-free buffer. Islets (200 per well, in triplicate) infected with Ad-eGFP or LtV-eGFP were washed extensively three days following infection, with Krebs-Ringer-HEPES buffer (KRH buffer; 25 mM HEPES pH 7.4, 115 mM NaCl, 24 mM NaHCO₃, 5 mM KCl, 2.5 mM CaCl₂, 1 mM MgCl₂) and incubated for 30 minutes at 37° C. Immediately thereafter, glucose was added to a final concentration of 5, 12 and 18 mM and the incubation carried out for a further 30 minutes. The buffer was collected and insulin detected using a commercially-available ELISA kit (CrystalChem, Chicago, Ill.).

### 8.2. Results

### 8.2.1. Comparison of Lentiviral and Adenoviral Gene Transfer to Human Islets in Vitro

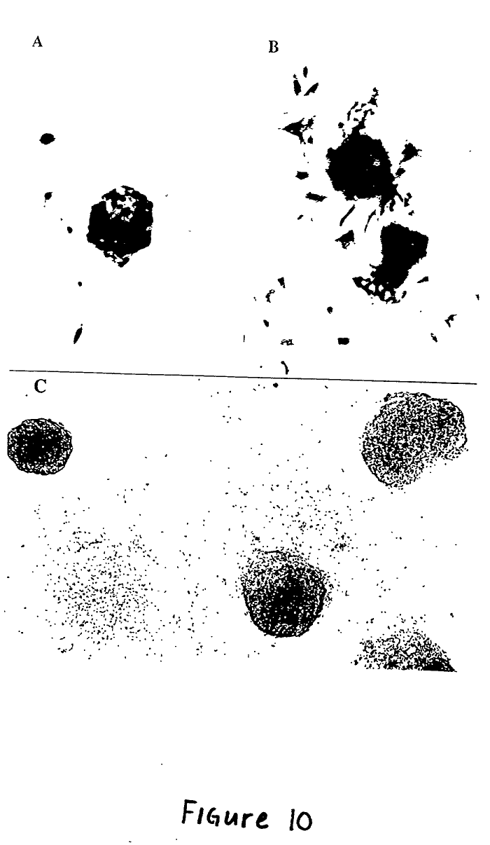

To evaluate the efficiency of infection by adenoviral and lentiviral vectors of intact human islets, equal plaque-forming units (pfu-adenovirus) or transducing-infectious units (TIU-lentivirus) of vectors carrying the β-galactosidase (LacZ) marker gene were used. Adenovirus (Ad)-LacZ infection was carried out for 4 hours and overnight with lentivirus (LtV)-LacZ. As shown in FIG. 10, both adenoviral and lentiviral vectors were able to infect the intact human islets with nearly identical efficiency (panels B and C are representative of the staining observed for all islets). There is no background β galactosidase expression in uninfected human islets (FIG. 10, Panel A).

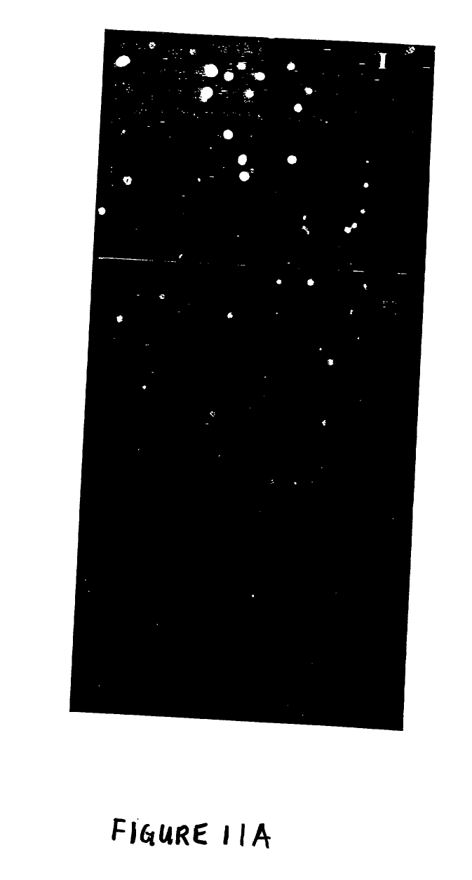

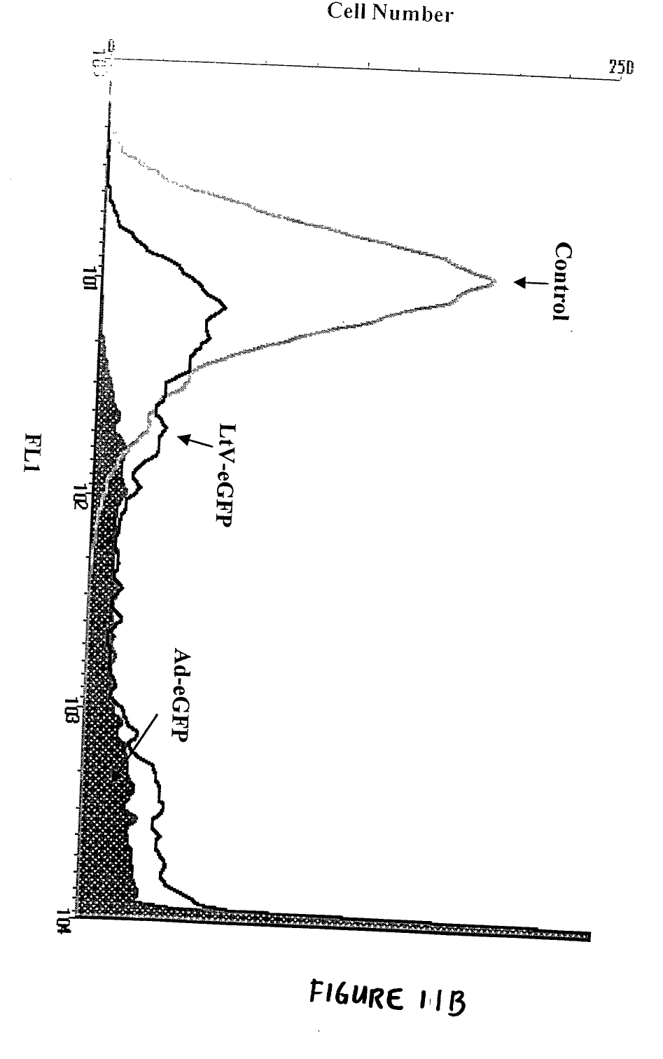

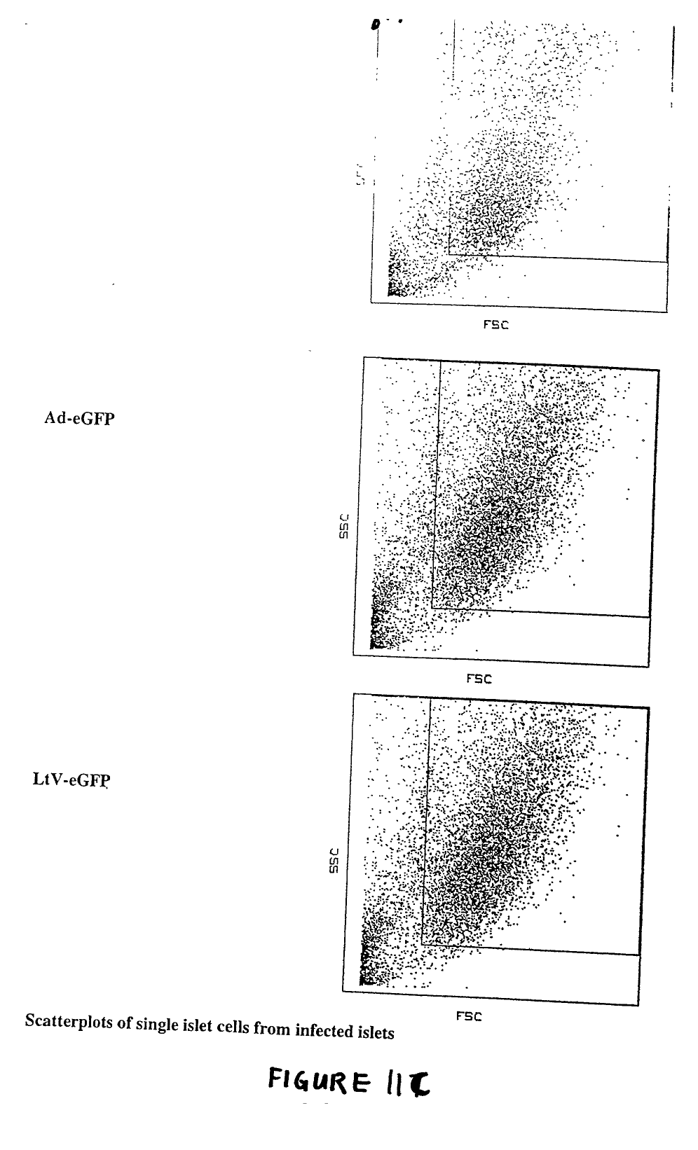

Since X-gal staining reveals only surface expression in intact islet and therefore could be misleading as to the true extent of gene transfer, single islet cells were analyzed by FACS following infection of intact islets with adenoviral and lentiviral vectors encoding the enhanced green fluorescent protein (eGFP). In FIG. 11A representative single islet cell populations expressing Ad-eGFP of LtV-eGF (Panel I: Ad-eGFP, Panel II: LtV-eGFP) are shown. Previous reports indicate that islet cells scatter between 1-15° forward scatter and that the predominant cell type in this peak is the β cell (Nielsen D. A. et al., 1982, Diabetes 31: 299-306). Thus the cells were gated on meeting this scatter profile and concurrently evaluated for eGFP fluorescence. Analysis of the eGFP positive cells indicated that the number of gated cells (i.e. β cells) expressing enhanced green fluorescent protein was almost identical irrespective of whether lentivirus or adenovirus was used as the transfer vehicle (FIG. 11B; LtV-eGFP : 51.6+/−0.8 vs. Ad-eGFP : 47.8+/−1.9; mean of three replicates +/− SEM). These results demonstrate that both the adenovirus and lentivirus vectors are able to efficiently infect β cells contained within intact islets. FACS analysis of dispersed single islet cells which are eGFP positive indicate that there is no significant difference between the number of gated cells that are eGFP positive derived from islets infected with an equal number of viral particles of LtV-eGFP or Ad-eGFP (FIG. 11C).

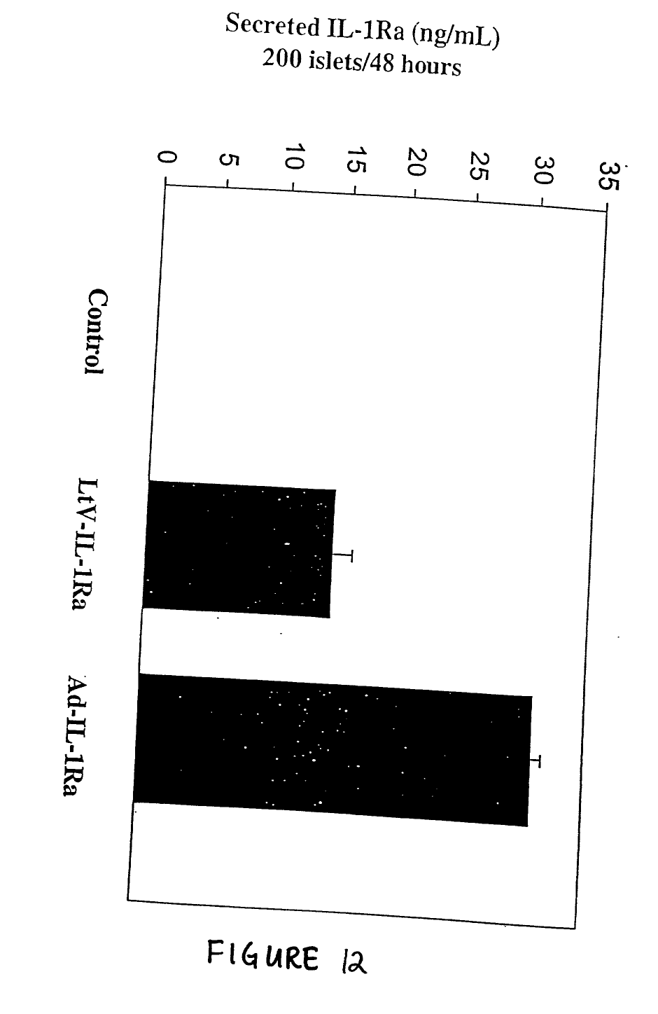

Interleukin 1β (IL-1β) is able to stimulate NO production in islets in vitro, resulting in islet dysfunction (Corbett J. A., 1993, Autoimmunity 15: 145-53; Corbett J. A. and McDaniel M. L., 1994, Biochem J 299: 719-24; Corbett J. A. and McDaniel M L, 1995, J Exp Med 181: 559-68). The IL-1β-dependent stimulation of NO also results in an increase in cell surface expression of Fas as well as increasing islet apoptosis in vitro in response to Fas triggering (Stassi G. et al., 1997, J Exp Med 186: 1193). In addition, the inhibition of IL-1β by continuous systemic administration of IL-1Ra has been shown to block islet dysfunction, insulitis onset and diabetes in NOD mice (Sandberg J. O. et al., 1997, Clin Exp Immunol 108: 314-7). Thus, stocks of lentiviral and adenoviral vectors expressing human interleukin-1 receptor antagonist protein were generated. Infection of 200 human islets with concentrated lentiviral IL-1Ra supernatant on two separate occasions yielded 13.5 and 15.1 ng/mL IL-1Ra by ELISA in the medium three days later. Similarly, Infection with Ad-IL-1Ra on two separate occasions led to levels of 29.4 and 32.6 ng/mL IL-1Ra, whereas mock-infected islets did not produce detectable IL-1Ra (FIG. 12).

### 8.2.2 Functional Evaluation of β Cells in Modified Intact Human Islets

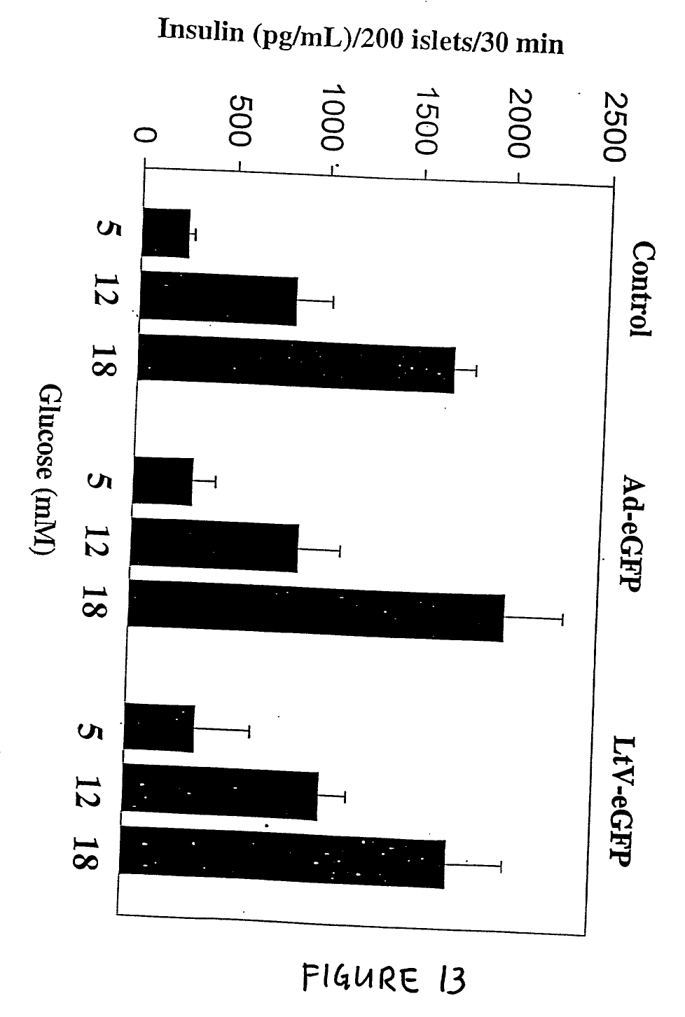

To determine if lentiviral infection and consequent transgene expression affect β cell function, the modified islets were challenged with increasing concentrations of glucose in a glucose-free buffer. FIG. 13 shows that insulin is secreted from virally-infected islets (adenoviral and lentiviral) into a serum-free buffer in a glucose concentration-dependent manner over a half-hour challenge in vitro. The profile of the LtV-eGFP (1×10⁶ TIU) response is identical to that of uninfected islets and islets infected with Ad-eGFP (1×10⁶ pfu). These results demonstrated that infection with lentiviral or first generation adenoviral vectors does not affect the function of β cells in vitro.

The present invention is not to be limited in scope by the embodiments disclosed in the examples which are intended as an illustration of one aspect of the invention, and any compositions or methods which are functionally equivalent are within the scope of this invention. Indeed, various modifications of the invention in addition to those shown and described herein will become apparent to those skilled in the art from the foregoing description. Such modifications are intended to fall within the scope of the claims.

Various references are cited herein, the disclosures of which are incorporated by reference in their entirites.

What is claimed:

1. A method for reducing β-cell dysfunction in an individual with a pancreatic disorder, comprising:
(a) introducing a nucleic acid molecule encoding an inhibitor of IL-1β into a β cell; and
(b) transplanting the β cell of step (a) into the idividual so as to reduce β cell dysfunction.

2. The method of claim 1 wherein the inhibitor of IL-1β activity is an interleukin-1 receptor antagonist protein.

3. The method of claim 1 wherein the inhibitor of IL-1β activity is an NF-κβ inhibitor.

4. The method of claim 1 wherein the inhibitor of IL-1β is an insulin like growth factor-1.

5. A method for reducing Fas mediated β-cell apoptosis in an individual with a pancreatic disorder, comprising:
(a) introducing a nucleic acid molecule encoding an inhibitor of Fas mediated apoptosis into a β cell; and
(b) transplanting the β cell of step (a) into the idividual so as to reduce β cell apoptosis.

6. The method of claim 5 wherein the inhibitor of Fas mediated apoptosis is an dominant negative mutant of the Fas protein.

7. The method of claim 5 wherein the inhibitor of Fas mediated apoptosis is a dominant negative mutant of the FADD protein.

8. The method of claim 5 wherein the inhibitor of Fas mediated apoptosis is a member of the bcl-2 protein family.

9. A mammalian β-cell comprising a recombinant nucleic acid molecule, said nucleic acid molecule comprising and expressing an inhibitor of IL-1β activity, wherein the expression of the inhibitor of IL-1β activity reduces β cell dysfunction.

10. The β-cell of claim 9 wherein the inhibitor of IL-1β activity is an interleukin-1 receptor antagonist protein.

11. The β-cell of claim 9 wherein the inhibitor of IL-1β is an NF-Kβ inhibitor protein.

12. The β-cell of claim 9 wherein the inhibitor of IL-1β is an insulin like growth factor-1 protein.

13. A recombinant viral vector comprising a nucleic acid molecule encoding an inhibitor of IL-1β activity.

14. The recombinant viral vector of claim 13 wherein the inhibitor of IL-1β is an interleukin-1 receptor antagonist protein.

15. The recombinant viral vector of claim 14 wherein the inhibitor of IL-1β is a NF- kβ inhibitor.

16. The recombinant viral vector of claim 14 wherein the inhibitor of IL-1β is an insulin like growth factor-1 protein.

17. The recombinant viral vector of claim 14 wherein the recombinant viral vector is an adenovirus vector.

18. The recombinant viral vector of claim 14 wherein the recombinant viral vector is a lentivirus vector.

19. The recombinant viral vector of claim 14 wherein the recombinant viral vector is a herpes simplex viral vector.
